(19) United States
(12) Patent Application Publication
(10) Pub. No.: US 2024/0301029 A1
(43) Pub. Date: Sep. 12, 2024

(54) METHODS AND COMPOSITIONS FOR PURIFICATION OF TRIMERIC FUSION PROTEINS

(71) Applicant: Sichuan Clover Biopharmaceuticals, Inc., Chengdu Sichuan (CN)

(72) Inventors: Peng LIANG, Chengdu Sichuan (CN); Joshua LIANG, Chengdu Sichuan (CN)

(73) Assignee: Sichuan Clover Biopharmaceuticals, Inc., Chengdu Sichuan (CN)

(21) Appl. No.: 18/022,140

(22) PCT Filed: Aug. 30, 2021
(86) PCT No.: PCT/CN2021/115245

§ 371 Date: Feb. 18, 2023

(30) Foreign Application Priority Data
Aug. 31, 2020 (WO) PCT/CN2020/112439
Apr. 13, 2021 (WO) PCT/CN2021/087055

(51) Int. Cl. C07K 14/705; C07K 1/22; C07K 14/005; C07K 14/78

(52) U.S. Cl. CPC C07K 14/7056; C07K 1/22; C07K 14/005; C07K 14/78; C07K 2319/30; C12N 2770/20051

(57) ABSTRACT

The present disclosure provides methods and compositions for affinity purification of proteins containing a C-terminal polypeptide sequence of a procollagen, such as trimeric fusion proteins. Also provided are polypeptides, fusion proteins, and recombinant polypeptides containing an Endo 180 ecto-domain or portion thereof capable of reversibly binding a C-terminal polypeptide sequence of a procollagen. Also provided are articles of manufacture, kits, and apparatus related to the Endo 180 ecto-domain polypeptides.

### CROSS-REFERENCE TO RELATED APPLICATIONS

This application claims priority to and the benefit of International Patent Application No. PCT/CN2021/087055, filed Apr. 13, 2021, and International Patent Application No. PCT/CN2020/112439, filed Aug. 31, 2020, the disclosures of which applications are incorporated herein by reference in their entireties for all purposes.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: P10892-PCT.210826.Sequence Listing_ST25.txt, date recorded: Aug. 25, 2021, size: 160 KB).

### FIELD

The present disclosure relates in some aspects to methods and compositions for affinity purification of proteins containing a C-terminal polypeptide sequence of a procollagen, such as trimeric fusion proteins. Also provided herein are polypeptides, fusion proteins, and recombinant polypeptides containing an ecto-domain of Endo180 or portion thereof capable of reversibly binding a C-terminal polypeptide sequence of a procollagen, e.g., forming a complex with a trimeric fusion protein comprising a C-terminal polypeptide sequence of a procollagen.

### BACKGROUND

Many major disease targets exist as membrane-associated trimers and/or require trimerization to become active. Fusion proteins containing a C-terminal polypeptide sequence of a procollagen are capable of spontaneous self-trimerization, allowing for trimerization of protein domains of interest without affecting the native amino acid sequence of the domains or the natural three dimensional trimeric structure. Trimeric fusion proteins containing C-terminal polypeptide sequences of a procollagen may be produced and secreted by cells, resulting in the need to purify the secreted trimeric fusion proteins. Methods of purification that allow for reversible selection of the trimeric fusion protein are therefore needed to isolate the trimeric fusion proteins from other proteins and debris in a sample, e.g., cell culture media. Provided herein are methods, compositions, articles of manufacture, kits, and apparatus that meet such and other needs.

### SUMMARY

In some aspects, disclosed herein is a method for purifying a trimeric fusion protein from a sample, comprising contacting a sample comprising a trimeric fusion protein with a soluble Endo180 polypeptide linked to a support. In some embodiments, the trimeric fusion protein comprises a heterologous polypeptide sequence fused to a C-terminal polypeptide sequence of a procollagen. In some embodiments, a first fusion polypeptide comprising the C-terminal polypeptide sequence is capable of forming inter-polypeptide bond(s) (e.g., disulfide bond(s)) with the C-terminal polypeptide sequence of a second fusion polypeptide. In some embodiments, a first, a second, and a third fusion polypeptides each comprising a C-terminal polypeptide sequence of a procollagen are capable of forming inter-polypeptide bond(s) (e.g., disulfide bond(s)) with one another at the C-terminal polypeptide sequence, thereby forming a trimeric fusion protein disclosed herein. In some embodiments, the C-terminal polypeptide sequence derived from a prodomain sequence of a procollagen (e.g., human procollagen) mediates the trimerization of molecules of a fusion polypeptide comprising the C-terminal polypeptide sequence. In some embodiments, the first, second, and third fusion polypeptides are identical in amino acid sequence. In some embodiments, any two or more of the first, second, and third fusion polypeptides can comprise different amino acid sequences. In some embodiments, a fusion polypeptide comprising a C-terminal polypeptide sequence of a procollagen is capable of trimerization via the C-terminal polypeptide sequence. In any of the embodiments herein, the trimeric fusion protein can comprise a heterologous polypeptide sequence fused to a C-terminal polypeptide sequence of a procollagen, and the C-terminal polypeptide sequence is capable of self trimerization and specifically binding to an Endo180 ecto-domain polypeptide. In any of the embodiments herein, the Endo180 ecto-domain polypeptide can be adsorbed, bound, attached, or otherwise immobilized to a support (e.g., a stationary phase in chromatography), whereby the trimeric fusion protein and the Endo180 ecto-domain polypeptide form a complex on the support. In some embodiments, the C-terminal polypeptide sequence is capable of self trimerization and specifically binding to the soluble Endo180 polypeptide, whereby the trimeric fusion protein and the soluble Endo180 polypeptide form a complex on the support. In any of the embodiments herein, the Endo180 ecto-domain polypeptide may lack the Endo180 D1 (CysR) sequence. In any of the embodiments herein, the support can comprise a porous solid (e.g., glass, silica, or alumina) that is packed into a glass or metal tube or that constitutes the walls of an open-tube capillary.

In some embodiments, the Endo180 ecto-domain polypeptide is a soluble Endo180 polypeptide specifically binds to the C-terminal polypeptide sequence in a fusion polypeptide disclosed herein. In some embodiments, the Endo180 ecto-domain polypeptide specifically binds to a trimeric structure formed by the C-terminal polypeptide sequence of a fusion polypeptide or protein. In some embodiments, the trimeric fusion protein and the Endo180 ecto-domain polypeptide form a complex on the support. In some embodiments, the method comprises removing unbound and/or nonspecifically bound molecules from the complex. In some embodiments, the method comprises dissociating the complex to release the trimeric fusion protein, thereby purifying the trimeric fusion protein from the sample. In some embodiments, disclosed herein are compositions and methods for purifying a stabilized trimeric form of a fusion protein comprising a trimerization domain derived from a prodomain sequence of a procollagen (e.g., human procollagen), or a population of trimeric fusion proteins each comprising a different fusion partner fused to the trimerization domain derived from the procollagen prodomain sequence.

In some embodiments, provided herein is a method for purifying a trimeric fusion protein from a sample, comprising: (a) contacting a sample comprising a trimeric fusion protein with a soluble Endo180 polypeptide linked to a support, wherein the trimeric fusion protein comprises a heterologous polypeptide sequence fused to a C-terminal polypeptide sequence of a procollagen, wherein the C-terminal polypeptide sequences form inter-polypeptide bonds (e.g., disulfide bonds), and, wherein the soluble Endo180 polypeptide specifically binds to the C-terminal polypeptide sequence and/or trimer thereof, whereby the trimeric fusion protein and the soluble Endo180 polypeptide form a complex on the support; (b) removing unbound and/or nonspecifically bound molecules from the complex; and (c) dissociating the complex to release the trimeric fusion protein, thereby purifying the trimeric fusion protein from the sample.

In some aspects, disclosed herein is a recombinant polypeptide comprising an Endo180 ecto-domain sequence comprising or consisting of a sequence of Endo180 Domain 6 (D6), a sequence of Endo180 Domain 7 (D7), a sequence of Endo180 Domain 8 (D8), a sequence of Endo180 Domain 9 (D9), and/or a sequence of Endo180 Domain 10 (D10) (e.g., one or more copies of D6, D7, D8, D9, D10, D1-6, D2-6, D3-6, D4-6, D5-6, D1-7, D2-7, D3-7, D4-7, D5-7, D6-7, D1-8, D2-8, D3-8, D4-8, D5-8, D6-8, D7-8, D1-9, D2-9, D3-9, D4-9, D5-9, D6-9, D7-9, D8-9, D1-10, D2-10, D3-10, D4-10, D5-10, D6-10, D7-10, D8-10, and/or D9-10). In some embodiments, the polypeptide lacks an Endo180 D1 sequence, an Endo180 D2 sequence, an Endo180 D3 sequence, an Endo180 D4 sequence, an Endo180 D5 sequence, an Endo180 D7 sequence, an Endo180 D8 sequence, an Endo180 D9 sequence, or an Endo180 D10 sequence, or any combination thereof.

In some aspects, disclosed herein is a recombinant polypeptide comprising an Endo180 ecto-domain sequence comprising or consisting of a sequence of Endo180 Domain 6 (D6), a sequence of Endo180 Domain 7 (D7), a sequence of Endo180 Domain 8 (D8), a sequence of Endo180 Domain 9 (D9), and/or a sequence of Endo180 Domain 10 (D10) (e.g., one or more copies of D6, D7, D8, D9, D10, D1-6, D2-6, D3-6, D4-6, D5-6, D1-7, D2-7, D3-7, D4-7, D5-7, D6-7, D1-8, D2-8, D3-8, D4-8, D5-8, D6-8, D7-8, D1-9, D2-9, D3-9, D4-9, D5-9, D6-9, D7-9, D8-9, D1-10, D2-10, D3-10, D4-10, D5-10, D6-10, D7-10, D8-10, and/or D9-10), wherein the Endo180 ecto-domain sequence is fused to a heterologous polypeptide sequence.

In some aspects, disclosed herein is a recombinant polypeptide comprising a sequence at least 70%, at least 75%, at least 80%, at least 85%, at least 90%, at least 95%, at least 99%, or 100% identical to SEQ ID NO: 1, 2, 3, 4, 43, 44, 45, or 46, or a fragment or variant thereof.

In some aspects, disclosed herein is a complex, comprising: a molecule comprising a heterologous polypeptide sequence linked to a C-terminal polypeptide sequence of a procollagen, and a binding partner comprising an Endo180 ecto-domain sequence comprising or consisting of a sequence of Endo180 Domain 6 (D6), a sequence of Endo180 Domain 7 (D7), a sequence of Endo180 Domain 8 (D8), a sequence of Endo180 Domain 9 (D9), and/or a sequence of Endo180 Domain 10 (D10) (e.g., one or more copies of D6, D7, D8, D9, D10, D1-6, D2-6, D3-6, D4-6, D5-6, D1-7, D2-7, D3-7, D4-7, D5-7, D6-7, D1-8, D2-8, D3-8, D4-8, D5-8, D6-8, D7-8, D1-9, D2-9, D3-9, D4-9, D5-9, D6-9, D7-9, D8-9, D1-10, D2-10, D3-10, D4-10, D5-10, D6-10, D7-10, D8-10, and/or D9-10), wherein all or a portion of the C-terminal polypeptide sequence binds to all or a portion of the Endo180 ecto-domain sequence.

### BRIEF DESCRIPTION OF THE DRAWINGS

FIGS. 1A-1B show the identification of functional domains of Endo180 that are responsible for binding to the C-terminal region of α1(I) collagen. FIG. 1A shows: a schematic illustration of the 10 structural domains (D1-10) of the extracellular region of Endo180 (left panel); exemplary Endo180 domains fused to an Fc region (fusion proteins: D1-Fc, D1-3-Fc, D1-4-Fc, D1-5-Fc, D1-6-Fc, D1-7-Fc, and D1-10-Fc) separated by non-reducing SDS-PAGE (right upper panel); binding of human placental alkaline phosphatase collagen fusion construct (AP-Coll) to the exemplary Endo180 fusion proteins determined by non-reducing SDS-PAGE (right middle panel) and AP as a negative control (right lower panel). FIG. 1B shows AP binding activity to the exemplary Endo180 fusion proteins quantified by optical density (OD405).

FIGS. 2A-2B show the identification of Endo180 domains for procollagen binding. FIG. 2A shows binding activity of a human placental alkaline phosphatase collagen fusion construct (AP-Coll) to exemplary Endo180 fusion proteins (D6-Fc and D1-10-Fc) determined by non-reducing SDS-PAGE. AP alone served as a negative control. FIG. 2B shows the binding activity of AP alone and AP-Coll to the exemplary Endo180 fusion proteins quantified by optical density (OD405).

FIGS. 3A-3B show high levels SDS-PAGE analysis of expression and Protein A affinity purification of exemplary Endo180 D1-10-Fc fusion proteins (FIG. 3A) and exemplary Endo180 D6-Fc fusion proteins (FIG. 3B) using MAbSelect SuRe Protein A resins (GE BioSciences). The Endo180-Fc fusion proteins were expressed as secreted proteins in serum-free culture. After binding to the Protein A resin, the Endo180-Fc fusion proteins were eluted with acetic acid buffer (pH 3.5).

FIG. 4 shows SDS-PAGE analysis of affinity purification of an exemplary trimeric fusion protein comprising TRAIL fused to a C-terminal polypeptide sequence of a procollagen (this trimeric form of TRAIL is referred to as TRAIL-Trimer™) from a serum-free cell culture media using an exemplary Endo180 D1-10-Fc fusion protein as an affinity tag covalently linked to CNBr activated resins. The exemplary TRAIL-Trimer™ fusion protein was expressed in GH-CHO cells as a secreted protein, and the serum-free cell culture medium containing TRAIL-Trimer™ was loaded onto Protein A Sepharose resins with D1-10-Fc pre-captured to the column via the high-affinity interaction between Protein A and Fc. After washing off any unbound contaminating proteins, the bound TRAIL-Trimer™ was purified to near homogeneity in a single step using 0.5 M-1.0 M NaCl in phosphate buffered saline, and analyzed by SDS-PAGE as indicated.

FIGS. 5A-5D show SDS-PAGE analysis of affinity purification of an exemplary trimeric fusion protein comprising a SARS-CoV-2 spike (S) protein sequence fused to a C-terminal polypeptide sequence of a procollagen (this stabilized trimeric form of S protein is referred to as S-Trimer™, a SARS-CoV-2 subunit vaccine candidate) from cell culture media using exemplary Endo180-Fc fusion proteins as affinity tags non-covalently bound to Protein A Sepharose resins. FIG. 5A shows purification of the exemplary trimeric fusion protein via exemplary D1-10-Fc (SEQ ID NO: 2) fusion proteins non-covalently bound to Protein A Sepharose resins. FIG. 5B shows purification of the exemplary trimeric fusion protein via exemplary D6-Fc fusion proteins non-covalently bound to Protein A Sepharose resins. FIGS. 5C and 5D show the purity of the partially cleaved S-Trimer™ fusion protein at the S1/S2 boundary by furin protease purified from either the exemplary D1-10-Fc or D6-Fc affinity tag, respectively, analyzed by Size-exclusion SEC-HPLC. For both exemplary purifications, the S-Trimer™ was expressed in GH-CHO cells as a secreted protein, and the serum-free cell culture medium containing the S-Trimer™ was loaded onto Protein A Sepharose resins pre-captured either with D6-Fc or D1-10-Fc to the column via the high-affinity interaction between Protein A and Fc. After washing off any unbound contaminating proteins, the bound S-Trimer™ was purified to near homogeneity in a single step using 0.5 M-1.0 M NaCl in phosphate buffered saline as elution buffer, and analyzed by SDS-PAGE as indicated.

FIG. 6 shows SDS-PAGE analysis of purification of another exemplary trimeric fusion protein (a SARS-Cov-2 Spike protein antigen with furin cleavage site mutation) via exemplary D6-Fc fusion protein non-covalently bound to Protein A Sepharose resins. This another exemplary S-Trimer™ fusion protein was expressed in GH-CHO cells as a secreted protein, and the serum-free cell culture medium containing S-Trimer™ was loaded onto Protein A Sepharose resins with D6-Fc pre-captured to the column via the high-affinity interaction between Protein A and Fc. After washing off any unbound contaminating proteins, the bound furin site mutant S-Trimer™ was purified to near homogeneity in a single step using 0.5 M-1.0 M NaCl in phosphate buffered saline, and analyzed by SDS-PAGE as indicated. Note, in comparison with that of native S-Trimer™, the purified furin site mutant from of S-Trimer™ did not show any S1 subunit cleaved.

### DETAILED DESCRIPTION

Provided herein are methods for purifying proteins containing procollagen, such as a C-terminal polypeptide sequence of a procollagen. In some aspects, the purification methods described herein are affinity purification techniques, where compositions capable of reversibly binding to a C-terminal polypeptide sequence of a procollagen are utilized. In some embodiments, the reversible binding or complexing of the composition with the C-terminal polypeptide sequence of a procollagen allows for the selection and purification of proteins containing a C-terminal polypeptide sequence of a procollagen from samples. In some embodiments, the composition is or includes an Endo180 polypeptide capable of reversibly binding to a C-terminal polypeptide sequence of a procollagen.

Endo180 is a collagen binding protein that was discovered independently by three different groups. The Endo180 protein was originally identified as an antigen that could bind to four different monoclonal antibodies that had been raised to identify novel human fibroblast cell surface receptors, and was shown to be a constitutively recycling cell surface protein (Isacke et al. (1990) Mol Cell Biol 10(6), 2606-2618). Endo180 also was found to form a trimolecular cell surface complex with urokinase plasminogen activator (uPA) and its receptor (uPAR) through chemical cross-linking studies and thus also is named urokinase plasminogen activator receptor associated protein (uPARAP) (Behrendt et al., (2000) J Biol Chem 275(3), 1993-2002).

A third group discovered that Endo180 encoded a novel macrophage mannose receptor type C lectin that was present in an EST database (Wu et al., (1996) J Biol Chem 271(35), 21323-21330). This cell surface protein has been identified as the fourth member of the mannose receptor family, which is also composed of the macrophage mannose receptor, the M-type phospholipase A2 receptor, and DEC-205. The four receptors are all type I transmembrane proteins with a highly conserved structure composed of an N-terminal cysteine-rich or ricin-type domain, a fibronectin type II domain (FNII), 8 or 10 C-type lectin-like domains (CTLDs), a single transmembrane domain, and a short cytoplasmic domain (East, L., and Isacke, C. M. (2002) Biochim Biophys Acta 1572(2-3), 364-386).

Endo180 has been shown to act as a collagen receptor by binding to gelatin, type I, type II, type IV, and type V collagens, and also mediates endocytosis and turnover of these extracellular matrix proteins. The FNII domain of Endo180 (which shares a highly conserved sequence with other collagen binding FNII domains, such as fibronectin, MMP-2, and MMP-9) appears to be required for binding to these collagens. It has been shown that mature native type I collagen binds to the first 4 domains of Endo180 (Paracuellos et al. (2015) Structure 23, 2133-2142). The region of collagen to which Endo180 binds has not been previously defined, due to the use of mature native collagens which lack any prodomains from tissue sources.

It has also been shown that Endo180 binds to a defined sequence of α1(I) collagen C-terminal region of collagen that includes C-prodomain (amino acids 1000-1453) (Thomas et al., J Biol Chem. (2005) 280(24), 22596-22605). To determine whether the α1(I) collagen C-terminal glycine repeat region or the C-prodomain alone were capable of initiating this binding, human placental secreted alkaline phosphatase (AP)-Tag technology was used to develop AP-Gly (amino acids 1000-1189) and AP-Pro (amino acids 1206-1453) fusion proteins, respectively (Thomas et al., J Biol Chem. (2005) 280(24), 22596-22605). However, these fusion proteins exhibited little binding to the surface of Rat-1 and Rat-1(Ras) cells compared to a human placental alkaline phosphatase collagen fusion construct (AP-Coll) containing the C-terminal glycine repeat region and prodomain of rat col1a1 (accession number Z78279). Although AP-Pro could form a trimeric structure, little binding was detected, suggesting that binding of Endo180 to soluble α1(I) collagen may require both a glycine repeat region and the C-prodomain or portions thereof.

The interaction between various collagen types and Endo180 appears to play a role in the cell-matrix adhesion, as Endo180-deficient cells have a 50% reduction in adhesion to type V collagen and also a reduced ability to bind to purified types I and IV collagen. To investigate whether the C-terminal glycine repeat region and C-prodomain of α1(I) collagen mediate this adhesion to Endo180, competition binding experiments were performed, and the results demonstrated that AP-Coll was able to significantly inhibit the adhesion of Rat-1 and Rat-1(Ras) cells specifically to type I collagen, suggesting that this region of collagen facilitates adhesion of cells to the extracellular matrix (Thomas et al., J Biol Chem. (2005) 280(24), 22596-22605).

Endo180 is a large type I transmembrane protein composed of 10 distinct structural domains that consist of an extracellular N-terminal cysteine-rich domain, a fibronectin type II domain (FNII), 8 consecutive C-type lectin-like domains (CTLDs), a single transmembrane domain, and a short cytoplasmic domain. To identify which structural domain(s) of Endo180 is involved in binding to C-terminal region of α1(I) collagen, structure-functional studies of its collagen binding site via serial deletions of Endo180 domains (D1-D10) were conducted as described in Examples 1-4 below. Domain 6 (D6) of the Endo180 ecto-domain, which is the 4th CTLD, was capable of procollagen binding (see, e.g., Example 2).

Examples 1 and 2 demonstrate the ability of the complete ecto-domain of Endo180 containing domains D1-10 or the D6 domain alone when fused to human IgG Fc could bind to a human placental alkaline phosphatase collagen fusion construct (AP-Coll) containing a C-terminal glycine repeat region and prodomain of rat col1a1 (accession number Z78279), and other recombinant proteins fused to the C-terminal glycine repeat region and prodomain of collagen, all with high affinity. Furthermore, as demonstrated in Examples 3 and 4, such binding affinity can be completely disrupted under moderate to high salt conditions.

Thus, in some aspects, the methods provided herein harness the discovery that the ecto-domain of Endo180, and specific domains thereof, bind to or complex with a C-prodomain of procollagen and a glycine repeat sequence of procollagen, e.g., as contained in a C-terminal polypeptide sequence of a procollagen, to create an affinity purification method capable of efficiently purifying proteins containing a C-terminal polypeptide sequence of a procollagen, including fusion proteins and recombinant proteins containing such sequences (Liu et al., (2017) Sci Rep. 7(1):8953).

In some embodiments, the methods provided herein include the use of Endo180 polypeptides, such as described in Section I-A, as affinity molecules or tags capable of complexing with, e.g., binding to, a C-terminal polypeptide sequence of a procollagen. The methods described herein are contemplated as useful for purifying any protein that contains procollagen, such as a C-terminal polypeptide sequence of a procollagen, including trimeric protein structures formed by C-terminal polypeptide sequences of procollagen. In some embodiments, the C-terminal polypeptide sequence of a procollagen, which includes the a C-prodomain of procollagen and a glycine repeat sequence of procollagen, to which the Endo180 polypeptide complexes is contained in a protein such as a fusion protein or recombinant polypeptide. In some embodiments, the fusion protein is a trimeric fusion protein, for example as described in Section II. In some embodiments, the trimeric fusion protein is a biologic drug.

Trimeric fusion proteins are of particular interest in treating diseases because many major disease targets exist as membrane-associated trimers and/or require trimerization to become active. Non-limiting examples of trimerization-dependent targets include the tumor necrosis factor (TNF) superfamily, which is involved in extrinsic apoptosis, immune co-stimulation, and inflammation, and enveloped RNA virus antigens responsible for entry into host cells. Fusion proteins containing C-terminal polypeptide sequences of a procollagen, which can include the C-prodomain of procollagen and a glycine repeat sequence of procollagen, are capable of spontaneous self-trimerization, allowing for trimerization of protein domains of interest without affecting the native amino acid sequence of the domains or the natural three dimensional trimeric structure formed by the native protein domains. Production of fusion proteins containing C-terminal polypeptide sequences of a procollagen to generate trimeric fusion proteins may be accomplished in vitro, for example by using cells to produce and secrete the trimeric fusion proteins into the cell culture media. Thus, the methods provided herein are particularly well-suited to purifying trimeric fusion proteins produced according to such methods.

In some aspects, the methods of purification provided herein utilize Endo180 polypeptides as described herein isolate proteins containing a C-terminal polypeptide sequence of a procollagen, e.g., trimeric fusion proteins, from samples containing mixtures of proteins and/or other molecules. In some embodiments, the sample is a biological sample. In some embodiments, the sample is a culture medium.

In some embodiments, proteins containing a C-terminal polypeptide sequence of a procollagen, e.g., trimeric fusion proteins, are purified from a sample by contacting the sample with an Endo180 polypeptide. In this way, the C-terminal polypeptide sequence of a procollagen can form a complex with the Endo180 polypeptide. In some embodiments, the Endo180 polypeptide is coupled to a support. Thus, in some cases, proteins containing a C-terminal polypeptide sequence of a procollagen, e.g., trimeric fusion proteins, are immobilized to a support via the Endo180 polypeptide. In some embodiments, immobilization of the proteins on the support allows unbound and/or nonspecifically bound molecules, e.g., from a mixed sample, to be separated from the protein of interest.

In some embodiments, the Endo180 polypeptide is covalently linked to a support, e.g., a solid support. In some embodiments, the Endo180 polypeptide is non-covalently linked to a support, e.g., a solid support. In some cases, the Endo180 polypeptide is a fusion protein including an affinity tag capable of non-covalently binding to a support, e.g., a solid support, having a cognate binding partner, e.g., receptor, of the affinity tag immobilized thereon. Thus, an Endo180 fusion protein containing an affinity tag can be loaded onto a support functionalized with the appropriate binding partner.

In some embodiments, the methods provided herein allow for efficient purification of proteins containing a C-terminal polypeptide sequence of a procollagen, e.g., trimeric fusion proteins, because the complex formed (e.g., bond) between the C-terminal polypeptide sequence of a procollagen of the protein and the Endo180 polypeptide is reversible. For example, in some embodiments, the complex can be dissociated in a single step by applying a buffer having a salt concentration to the support. Thus, in some embodiments, after removal of unbound proteins, molecules, and debris, the complex, e.g., binding, between the Endo180 polypeptide and the C-terminal polypeptide sequence of a procollagen, is dissociated, thereby releasing the isolated proteins from the support and resulting in a protein purified from the sample.

In some embodiments, dissociation of the complex takes place in a single step. In some embodiments, the dissociation is accomplished by addition of a buffer to the support. In some embodiments, the dissociation is accomplished by addition of a buffer containing a mild salt concentration to the solid support. In some embodiments, the dissociation is accomplished by addition of a buffer containing a moderate salt concentration to the solid support. In some embodiments, the dissociation is accomplished by addition of a buffer containing a high salt concentration to the solid support. In some embodiments, the salt concentration is between or between about 0.5 M-1.0 M. In some embodiments, the salt is NaCl.

The methods described herein provide an efficient and effective means of purifying proteins of interest containing a C-terminal polypeptide sequence of a procollagen. In some embodiments, the single step reversibility of complex formation between the C-terminal polypeptide sequence of a procollagen and the Endo180 polypeptide provides an ideal method for quickly purifying proteins, such as biologic drugs (e.g., trimeric fusion proteins), produced in vitro.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Methods for Affinity Purification

The methods provided herein allow for affinity purification of proteins containing a C-terminal polypeptide sequence of a procollagen. In some embodiments, the affinity purification methods are accomplished using an Endo180 polypeptide, for example as described in Section I-A below, capable of binding to a C-terminal polypeptide sequence of a procollagen.

In some embodiments, the Endo180 polypeptide forms a reversible complex with, e.g., binds to, a C-terminal polypeptide sequence of procollagen which includes a C-prodomain of a procollagen and a glycine repeat sequence of a procollagen or portions thereof. Thus, in some embodiments, the methods allow for selection and isolation of proteins of interest containing a C-terminal polypeptide sequence of procollagen from a sample, e.g., a mixed sample, and elution and collection of the protein of interest of by dissociating the complex.

In some embodiments, the Endo180 polypeptide is linked to a support, for example a support described in Section I-B. In some embodiments, the Endo180 polypeptide is linked to a solid support. In some embodiments, the support is a resin, a particle, a bead, a solid substrate, a membrane, or a combination thereof. Thus, in some cases, the protein of interest containing the C-terminal polypeptide sequence of a procollagen is immobilized on the support via the Endo180 polypeptide. In some embodiments, immobilization of the protein of interest on the support allows for the protein of interest to be separated from other proteins and/or debris. In some embodiments, unbound and/or nonspecifically bound molecules are removed from the complex using a wash step. In some embodiments, one or more wash steps may be used to remove unbound or nonspecifically bound molecules from the support. In some embodiments, the wash step(s) involves introducing a buffer to the support.

In some embodiments, the complex formed between the immobilized protein and the Endo180 polypeptide is dissociated by addition of a buffer. In some embodiments, the buffer contains a salt concentration capable of dissociating the complex, e.g., bond, formed between the C-terminal polypeptide sequence of a procollagen contained in the protein of interest and the Endo180 polypeptide. In some embodiments, the buffer contains a salt concentration between or between about 0.1 M to about 1.5 M, about 0.1 M to about 1.4 M, about 0.1 M to about 1.3 M, about 0.1 M to about 1.2 M, about 0.1 M to about 1.1 M, about 0.1 M to about 1.0 M, about 0.1 M to about 0.9 M, about 0.1 M to about 0.8 M, about 0.1 M to about 0.7 M, about 0.1 M to about 0.6 M, about 0.1 M to about 0.5 M, about 0.1 M to about 0.4 M, about 0.1 M to about 0.3 M, about 0.1 M to about 0.2 M, about 0.2 M to about 1.5 M, about 0.3 M to about 1.5 M, about 0.4 M to about 1.5 M, about 0.4 M to about 1.5 M, about 0.5 M to about 1.5 M, about 0.6 M to about 1.5 M, about 0.7 M to about 1.5 M, about 0.8 M to about 1.5 M, about 0.9 M to about 1.5 M, about 1.0 M to about 1.5 M, about 1.1 M to about 1.5 M, about 1.2 M to about 1.5 M, about 1.3 M to about 1.5 M, or about 1.4 M to about 1.5 M, inclusive. In some embodiments, the salt concentration is between or between about 0.5 M to about 1.0 M, inclusive. In some embodiments, the salt is NaCl.

In some embodiments, the complex between the immobilized protein and the Endo180 polypeptide is dissociated in a single step, e.g., by addition of buffer once to the support. In some embodiments, the complex between the immobilized protein and the Endo180 polypeptide is dissociated in two or more steps, e.g., including multiple additions of buffer to the support. In some embodiments, for example when two or more steps are used for dissociating the complex, one or more buffers with differing salt concentrations are used to dissociate the complex. In some embodiments, a plurality of buffers having increasing salt concentrations are used in a plurality of steps to dissociate the complex.

In some embodiments, dissociating the complex formed between the immobilized protein and the Endo180 polypeptide frees the protein of interest from the support, allowing elution of the purified protein of interest. In some embodiments, the protein of interest is collected, e.g., in a container (see, e.g., Section III). In some embodiments, the eluted protein of interest is further processed. For example, in some embodiments, if the purified protein of interest is a therapeutic, e.g., biologic, the protein can be formulated for administration, e.g., formulated as a pharmaceutical composition.

In some embodiments, the protein of interest is a trimeric fusion protein containing a C-terminal polypeptide sequence of a procollagen. In some embodiments, the trimeric fusion protein includes a heterologous polypeptide sequence fused to a C-terminal polypeptide sequence of a procollagen. In some embodiments, the C-terminal polypeptide sequence is capable of self trimerization, thereby generating the trimeric fusion protein.

Many major disease targets exist as membrane-associated trimers and/or require trimerization to become active. Fusion proteins containing a C-terminal polypeptide sequence of a procollagen are capable of spontaneous self-trimerization through the C-terminal polypeptide sequence of a procollagen, which allows for trimerization of protein of interest without affecting the native amino acid sequence of the domains. Production of such trimeric fusion proteins may be accomplished in vitro, for example by infecting cells with polynucleotides encoding fusion proteins, which once expressed trimerize to form trimeric fusion proteins that can be secreted from the cell. Such production necessitates purification of the secreted trimeric fusion proteins from the cell culture media. The methods of purification provided herein are suitable for purification of trimeric fusion proteins, such as described in Section II.

### A. Endo180 Polypeptides

The methods described herein include the use of Endo180 polypeptides, e.g., one or more Endo180 ecto-domain polypeptide, such as polypeptide comprising or consisting of all or a portion of the Endo180 Domain 6 (D6) sequence. In some embodiments, the Endo180 ecto-domain polypeptide comprises an Endo180 ecto-domain sequence comprising or consisting of a sequence of Endo180 Domain 6 (D6), a sequence of Endo180 Domain 7 (D7), a sequence of Endo180 Domain 8 (D8), a sequence of Endo180 Domain 9 (D9), and/or a sequence of Endo180 Domain 10 (D10). In some embodiments, the Endo180 ecto-domain polypeptide comprises an Endo180 ecto-domain sequence comprising or consisting of one or more copies of D6, D7, D8, D9, D10, D1-6, D2-6, D3-6, D4-6, D5-6, D1-7, D2-7, D3-7, D4-7, D5-7, D6-7, D1-8, D2-8, D3-8, D4-8, D5-8, D6-8, D7-8, D1-9, D2-9, D3-9, D4-9, D5-9, D6-9, D7-9, D8-9, D1-10, D2-10, D3-10, D4-10, D5-10, D6-10, D7-10, D8-10, and/or D9-10, or a fragment or variant thereof.

In some aspects, the Endo180 polypeptides described herein are capable of complexing, e.g., binding, to a C-terminal polypeptide sequence of a procollagen. In some embodiments, the Endo180 polypeptide provided herein is mammalian, optionally human. The term Endo180 polypeptide as used herein refers to any Endo180 polypeptide, Endo180 fusion protein, Endo180 recombinant polypeptide, Endo180 containing binding partner, or Endo180 containing binding reagent described herein, unless otherwise stated.

In some embodiments, the Endo180 polypeptide is soluble. For example, in some cases, the Endo180 polypeptide does not include a transmembrane domain and/or a cytoplasmic domain. In some embodiments, the soluble Endo180 polypeptide is or includes an ecto-domain of an Endo180 protein. In some embodiments, the Endo180 ecto-domain is or includes the sequence set forth in SEQ ID NO: 7. In some embodiments, the Endo180 ecto-domain is or includes a sequence with at least 75%, 80%, 85%, 90%, 91%, 92%, 93%, 94%, 95%, 96%, 97%, 98%, or 99% sequence identity with the sequence set forth in SEQ ID NO: 7. In some embodiments, the soluble Endo180 polypeptide is or includes one or more domains contained in the Endo180 ecto-domain.

The ecto-domain of Endo180 is composed of 10 distinct structural domains that consist of an extracellular N-terminal cysteine-rich domain, referred to herein as D1; a fibronectin type II domain (FNII), referred to herein as D2; and 8 consecutive C-type lectin-like domains (CTLDs), referred to herein as D1-D8, respectively. In some embodiments, the sequences of D1-D10 are or include the sequence set forth in SEQ ID NOS: 8-17, respectively. In some embodiments, the sequences of D1-D10 are or include a sequence with at least 75%, 80%, 85%, 90%, 91%, 92%, 93%, 94%, 95%, 96%, 97%, 98%, or 99% sequence identity with the sequences set forth in SEQ ID NOS: 8-17, respectively.

In some embodiments, the soluble Endo180 polypeptide includes domains 1-10 (D1-10) of Endo180. In some embodiments, the soluble Endo180 polypeptide includes domains 6-10 (D6-10) of Endo180. In some embodiments, the soluble Endo180 polypeptide includes domains 6-9 (D6-9) of Endo180. In some embodiments, the soluble Endo180 polypeptide includes domains 6-8 (D6-8) of Endo180. In some embodiments, the soluble Endo180 polypeptide includes domains 6-7 (D6-7) of Endo180. In some embodiments, the soluble Endo180 polypeptide includes domains 6-10 (D6-10) of Endo180. In some embodiments, the soluble Endo180 polypeptide incudes domain 6 (D6) of Endo180.

In some embodiments, the soluble Endo180 polypeptide incudes two or more domain 6 (D6) of Endo180. In some embodiments, the soluble Endo180 polypeptide incudes two or more consecutive domain 6 (D6) of Endo180. In some embodiments, the soluble Endo180 polypeptide incudes three domain 6 (D6) of Endo180.

In some embodiments, the soluble Endo180 polypeptide does not include any one or more of domains 1-5 (D1-5) of Endo180. In some embodiments, the soluble Endo180 polypeptide does not include any one or more of domains 1-4 (D1-4) of Endo180.

In some embodiments, the soluble Endo180 polypeptide includes domains 1-6 (D1-6) of Endo180. In some embodiments, the soluble Endo180 polypeptide includes domains 2-6 (D2-6) of Endo180. In some embodiments, the soluble Endo180 polypeptide includes domains 3-6 (D3-6) of Endo180. In some embodiments, the soluble Endo180 polypeptide includes domains 4-6 (D4-6) of Endo180. In some embodiments, the soluble Endo180 polypeptide includes domains 5-6 (D5-6) of Endo180.

In some embodiments, the soluble Endo180 polypeptide is a fusion protein. In some embodiments, the Endo180 fusion protein includes a heterologous polypeptide which may include any polypeptide that is not Endo180 or a fragment or variant thereof.

In some embodiments, the heterologous polypeptide sequence includes a tag, such as a tag for affinity purification. For example, in some cases, the Endo180 polypeptide is linked to a support, such as a solid support, such that the complexing between the C-terminal polypeptide sequence of a procollagen contained in the protein of interest the Endo180 polypeptide results in the immobilization of the protein of interest on the support. In some cases, the Endo180 polypeptide is linked directly to the support, e.g., via a covalent linkage. Alternatively, for example when the Endo180 polypeptide is a fusion protein, the Endo180 polypeptide is linked to the support non-covalently, optionally through binding of an affinity tag contained in the Endo180 fusion protein to a cognate binding partner immobilized on the support. In some embodiments, when the Endo180 polypeptide is linked to a support, the Endo180 polypeptide is no longer considered soluble.

It is contemplated that any suitable affinity tag may be included in the Endo180 fusion protein. In some embodiments, the affinity tag included in the Endo180 fusion protein is biotin. In some embodiments, the biotin binds to streptavidin immobilized on the support. In some embodiments, the biotin binds to avidin immobilized on the support.

In some embodiments, the affinity tag included in the Endo180 fusion protein is an Fc region. In some embodiments, the soluble Endo180 is an Endo180-Fc fusion protein. In some embodiments, the Fc region is a human Fc region. In some embodiments, the Fc region is an IgG Fc region. In some embodiments, the Fc region is or includes the sequence set forth by SEQ ID NO: 6 or SEQ ID NO: 42. In some embodiments, the Fc region is or includes a sequence at least 70%, at least 75%, at least 80%, at least 85%, at least 90%, at least 95%, at least 99%, or 100% identical to SEQ ID NO: 6 or SEQ ID NO: 42. In some embodiments, the Fc region binds non-covalently to Protein A immobilized on a support. In some embodiments, the support is a Protein A resin. In some embodiments, the Protein A resin is Sepharose.

In some embodiment, the soluble Endo180 polypeptide is a D1-10-Fc fusion protein. In some embodiments, the soluble Endo180 polypeptide includes a sequence at least 70%, at least 75%, at least 80%, at least 85%, at least 90%, at least 95%, at least 99%, or 100% identical to SEQ ID NO: 1 or SEQ ID NO: 2, or a fragment or variant thereof. In some embodiments, the soluble Endo180 polypeptide includes a sequence at least 70%, at least 75%, at least 80%, at least 85%, at least 90%, at least 95%, at least 99%, or 100% identical to SEQ ID NO: 43 or SEQ ID NO: 44, or a fragment or variant thereof.

In some embodiments, the soluble Endo180 polypeptide is a D6-10-Fc fusion protein. In some embodiments, the soluble Endo180 polypeptide is a D6-9-Fc fusion protein. In some embodiments, the soluble Endo180 polypeptide is a D6-8-Fc fusion protein. In some embodiments, the soluble Endo180 polypeptide is a D6-7-Fc fusion protein.

In some embodiments, the soluble Endo180 polypeptide is a D6-Fc fusion protein. In some embodiments, the soluble Endo180 polypeptide comprises a sequence at least 70%, at least 75%, at least 80%, at least 85%, at least 90%, at least 95%, at least 99%, or 100% identical to SEQ ID NO: 3 or SEQ ID NO: 4, or a fragment or variant thereof. In some embodiments, the soluble Endo180 polypeptide comprises a sequence at least 70%, at least 75%, at least 80%, at least 85%, at least 90%, at least 95%, at least 99%, or 100% identical to SEQ ID NO: 45 or SEQ ID NO: 46, or a fragment or variant thereof.

In some embodiments, the soluble Endo180 polypeptide is a (D6)₂-Fc fusion protein. In some embodiments, the soluble Endo180 polypeptide is a (D6)₃-Fc fusion protein. In some embodiments, the soluble Endo180 polypeptide is a (D6)ₙ-Fc fusion protein, wherein n is an integer of 4, 5, 6, 7, 8, 9, 10, or more. In some embodiments, the soluble Endo180 polypeptide is a D1-6-Fc fusion protein. In some embodiments, the soluble Endo180 polypeptide is a D2-6-Fc fusion protein. In some embodiments, the soluble Endo180 polypeptide is a D3-6-Fc fusion protein. In some embodiments, the soluble Endo180 polypeptide is a D4-6-Fc fusion protein. In some embodiments, the soluble Endo180 polypeptide is a D5-6-Fc fusion protein.

In some embodiments, the Endo180 polypeptide is contained in a binding reagent. In some embodiments, the binding reagent includes an Endo180 ecto-domain sequence including an Endo180 Domain 6 (D6) sequence that is capable of complexing with a target molecule including a C-terminal polypeptide sequence of a procollagen, wherein the complex is formed between all or a portion of the C-terminal polypeptide sequence and all or a portion of the Endo180 Domain 6 (D6) sequence. In some embodiments, the binding reagent lacks any one or more of Endo180 D1, D2, D3, D4, D5, D7, D8, D9, and D10. In some embodiments, the binding reagent lacks an Endo180 Domain 1 (D1) sequence. In some embodiments, the binding reagent lacks an Endo180 Domain 2 (D2) sequence. In some embodiments, the binding reagent lacks an Endo180 Domain 3 (D3) sequence. In some embodiments, the binding reagent lacks an Endo180 Domain 4 (D4) sequence. In some embodiments, the binding reagent lacks an Endo180 Domain 5 (D5) sequence. In some embodiments, the binding reagent lacks an Endo180 Domain 7 (D7) sequence. In some embodiments, the binding reagent lacks an Endo180 Domain 8 (D8) sequence. In some embodiments, the binding reagent lacks an Endo180 Domain 9 (D9) sequence. In some embodiments, the binding reagent lacks an Endo180 Domain 10 (D10) sequence.

In some embodiments, the binding reagent includes a fusion polypeptide containing Endo180 Domains 1-6 (D1-6), D2-6, D3-6, D4-6, D5-6, one or more D6, D6-7, D6-8, D6-9, or D6-10, or a combination thereof. In some embodiments, the binding reagent includes two or more D6. In some embodiments, the binding reagent includes two or more consecutive D6.

In any of the embodiments herein, the binding reagent can comprise an Endo180 ecto-domain sequence comprising or consisting of a sequence of Endo180 Domain 6 (D6), a sequence of Endo180 Domain 7 (D7), a sequence of Endo180 Domain 8 (D8), a sequence of Endo180 Domain 9 (D9), and/or a sequence of Endo180 Domain 10 (D10). In any of the embodiments herein, the binding reagent can comprise an Endo180 ecto-domain sequence comprising or consisting of one or more copies of D6, D7, D8, D9, D10, D1-6, D2-6, D3-6, D4-6, D5-6, D1-7, D2-7, D3-7, D4-7, D5-7, D6-7, D1-8, D2-8, D3-8, D4-8, D5-8, D6-8, D7-8, D1-9, D2-9, D3-9, D4-9, D5-9, D6-9, D7-9, D8-9, D1-10, D2-10, D3-10, D4-10, D5-10, D6-10, D7-10, D8-10, and/or D9-10, in any suitable combination.

In some embodiments, the binding reagent is soluble. In some embodiments, the binding reagent is linked to a support, for examples as described in Section I-B. In some embodiments, the binding reagent is covalently linked to the support. In some embodiments, the bind reagent is non-covalently linked to the support. For example, when the binding reagent includes a fusion protein, the fusion protein may contain an affinity tag, such as described herein, which facilitates non-covalent binding between the binding reagent and a cognate binding partner immobilized on the support.

In some embodiments, the Endo180 polypeptide is a recombinant polypeptide. In some embodiments, the recombinant polypeptide includes an Endo180 Domain 6 (D6) sequence, and the recombinant polypeptide lacks an Endo180 D1 sequence, an Endo180 D2 sequence, an Endo180 D3 sequence, an Endo180 D4 sequence, an Endo180 D5 sequence, an Endo180 D7 sequence, an Endo180 D8 sequence, an Endo180 D9 sequence, or an Endo180 D10 sequence, or any combination thereof. In some embodiments, the recombinant polypeptide lacks one or more of Endo180 D1, D2, D3, D4, D5, D7, D8, D9, and D10. In some embodiments, the recombinant polypeptide consists of Endo180 Domains 1-6 (D1-6), D2-6, D3-6, D4-6, D5-6, one or more D6, D6-7, D6-8, D6-9, or D6-10, or a combination thereof. In some embodiments, the recombinant polypeptide consists of Endo180 Domains 1-7 (D1-7), D2-7, D3-7, D4-7, D5-7, one or more D6, D6-7, D6-8, D6-9, or D6-10, or a combination thereof. In some embodiments, the recombinant polypeptide consists of Endo180 Domains 1-8 (D1-8), D2-8, D3-8, D4-8, D5-8, one or more D6, D6-7, D6-8, D6-9, or D6-10, or a combination thereof. In some embodiments, the recombinant polypeptide consists of Endo180 Domains 1-9 (D1-9), D2-9, D3-9, D4-9, D5-9, one or more D6, D6-7, D6-8, D6-9, or D6-10, or a combination thereof. In some embodiments, the recombinant polypeptide consists of Endo180 Domains 1-10 (D1-10), D2-10, D3-10, D4-10, D5-10, one or more D6, D6-7, D6-8, D6-9, or D6-10, or a combination thereof. In some embodiments, the recombinant polypeptide includes two or more D6. In some embodiments, the recombinant polypeptide includes two or more consecutive D6.

In some embodiments, provided herein is a polypeptide or complex thereof comprising an Endo180 Domain 6 (D6) moiety and a moiety of interest, and the polypeptide or complex lacks an Endo180 D1 sequence, an Endo180 D2 sequence, an Endo180 D3 sequence, an Endo180 D4 sequence, an Endo180 D5 sequence, an Endo180 D7 sequence, an Endo180 D8 sequence, an Endo180 D9 sequence, or an Endo180 D10 sequence, or any combination thereof. In some embodiments, the polypeptide or complex lacks one or more of Endo180 D1, D2, D3, D4, D5, D7, D8, D9, and D10. In some embodiments, the polypeptide or complex comprises Endo180 Domains 1-6 (D1-6), D2-6, D3-6, D4-6, D5-6, one or more D6, D6-7, D6-8, D6-9, or D6-10, or a combination thereof, and a moiety of interest. In some embodiments, the polypeptide or complex comprises Endo180 Domains 1-7 (D1-7), D2-7, D3-7, D4-7, D5-7, one or more D6, D6-7, D6-8, D6-9, or D6-10, or a combination thereof, and a moiety of interest. In some embodiments, the polypeptide or complex comprises Endo180 Domains 1-8 (D1-8), D2-8, D3-8, D4-8, D5-8, one or more D6, D6-7, D6-8, D6-9, or D6-10, or a combination thereof, and a moiety of interest. In some embodiments, the polypeptide or complex comprises Endo180 Domains 1-9 (D1-9), D2-9, D3-9, D4-9, D5-9, one or more D6, D6-7, D6-8, D6-9, or D6-10, or a combination thereof, and a moiety of interest. In some embodiments, the polypeptide or complex comprises Endo180 Domains 1-10 (D1-10), D2-10, D3-10, D4-10, D5-10, one or more D6, D6-7, D6-8, D6-9, or D6-10, or a combination thereof, and a moiety of interest. In some embodiments, the polypeptide or complex comprises two or more D6 and a moiety of interest. In some embodiments, the polypeptide or complex comprises two or more consecutive D6 and a moiety of interest.

In some embodiments, the recombinant polypeptide includes an Endo180 ecto-domain sequence including an Endo180 Domain 6 (D6) sequence, wherein the Endo180 ecto-domain sequence is fused to a heterologous polypeptide sequence which may include any polypeptide that is not Endo180 or a fragment or variant thereof.

In some embodiments, the heterologous polypeptide sequence includes a tag, e.g., a tag for affinity purification, such as described above. In some embodiments, the affinity tag is biotin or an Fe region. In some embodiments, the recombinant polypeptide includes biotin and is capable of binding to streptavidin or avidin immobilized on a support as described herein. In some embodiments, the recombinant polypeptide includes an Fc region and is capable of binding to Protein A immobilized in a support as described herein. In some embodiments, the Fc region is or includes the sequence set forth by SEQ ID NO: 6. In some embodiments, the Fc region is or includes a sequence at least 70%, at least 75%, at least 80%, at least 85%, at least 90%, at least 95%, at least 99%, or 100% identical to SEQ ID NO: 6. In some embodiments, the recombinant polypeptide includes Endo180 Domains 1-6 (D1-6), D2-6, D3-6, D4-6, D5-6, one or more D6, D6-7, D6-8, D6-9, or D6-10, or a combination thereof. In some embodiments, the recombinant polypeptide includes Endo180 Domains 1-6 fused to an Fc region (D1-6-Fc), D2-6-Fc, D3-6-Fc, D4-6-Fc, D5-6-Fc, (D6)ₙ-Fc, D6-7-Fc, D6-8-Fc, D6-9-Fc, D6-10-Fc, D1-10-Fc, or a combination thereof, wherein n is an integer of 1 or greater.

In some embodiments, the recombinant polypeptide includes a sequence at least 70%, at least 75%, at least 80%, at least 85%, at least 90%, at least 95%, at least 99%, or 100% identical to SEQ ID NO: 1, 2, 3, or 4 or a fragment or variant thereof. In some embodiments, the recombinant polypeptide comprises the sequence of SEQ ID NO: 1. In some embodiments, the recombinant polypeptide includes a sequence at least 70%, at least 75%, at least 80%, at least 85%, at least 90%, at least 95%, at least 99%, or 100% identical to SEQ ID NO: 1. In some embodiments, the recombinant polypeptide comprises the sequence of SEQ ID NO: 2. In some embodiments, the recombinant polypeptide includes a sequence at least 70%, at least 75%, at least 80%, at least 85%, at least 90%, at least 95%, at least 99%, or 100% identical to SEQ ID NO: 2. In some embodiments, the recombinant polypeptide comprises the sequence of SEQ ID NO: 3. In some embodiments, the recombinant polypeptide includes a sequence at least 70%, at least 75%, at least 80%, at least 85%, at least 90%, at least 95%, at least 99%, or 100% identical to SEQ ID NO: 3. In some embodiments, the recombinant polypeptide comprises the sequence of SEQ ID NO: 4. In some embodiments, the recombinant polypeptide includes a sequence at least 70%, at least 75%, at least 80%, at least 85%, at least 90%, at least 95%, at least 99%, or 100% identical to SEQ ID NO: 4.

In some embodiments, the recombinant polypeptide includes a sequence at least 70%, at least 75%, at least 80%, at least 85%, at least 90%, at least 95%, at least 99%, or 100% identical to SEQ ID NO: 43, 44, 45, or 46 or a fragment or variant thereof. In some embodiments, the recombinant polypeptide comprises the sequence of SEQ ID NO: 43. In some embodiments, the recombinant polypeptide includes a sequence at least 70%, at least 75%, at least 80%, at least 85%, at least 90%, at least 95%, at least 99%, or 100% identical to SEQ ID NO: 43. In some embodiments, the recombinant polypeptide comprises the sequence of SEQ ID NO: 44. In some embodiments, the recombinant polypeptide includes a sequence at least 70%, at least 75%, at least 80%, at least 85%, at least 90%, at least 95%, at least 99%, or 100% identical to SEQ ID NO: 44. In some embodiments, the recombinant polypeptide comprises the sequence of SEQ ID NO: 45. In some embodiments, the recombinant polypeptide includes a sequence at least 70%, at least 75%, at least 80%, at least 85%, at least 90%, at least 95%, at least 99%, or 100% identical to SEQ ID NO: 45. In some embodiments, the recombinant polypeptide comprises the sequence of SEQ ID NO: 46. In some embodiments, the recombinant polypeptide includes a sequence at least 70%, at least 75%, at least 80%, at least 85%, at least 90%, at least 95%, at least 99%, or 100% identical to SEQ ID NO: 46.

In some embodiments, the Endo180 polypeptide is contained in a binding partner. In some embodiments, the binding partner includes an Endo180 ecto-domain sequence comprising an Endo180 Domain 6 (D6) sequence capable of binding to a molecules including a heterologous polypeptide sequence linked to a C-terminal polypeptide sequence of a procollagen, wherein all or a portion of the C-terminal polypeptide sequence binds to all or a portion of the Endo180 Domain 6 (D6) sequence. In some embodiments, the binding partner includes Endo180 Domains 1-6 fused to an Fc region (D1-6-Fc), D2-6-Fc, D3-6-Fc, D4-6-Fc, D5-6-Fc, (D6)ₙ-Fc, D6-7-Fc, D6-8-Fc, D6-9-Fc, D6-10-Fc, D1-10-Fc, or a combination thereof, wherein n is an integer of 1 or greater. In some embodiments, the Fc region is or includes the sequence set forth by SEQ ID NO: 6. In some embodiments, the Fc region is or includes a sequence at least 70%, at least 75%, at least 80%, at least 85%, at least 90%, at least 95%, at least 99%, or 100% identical to SEQ ID NO: 6 or 42. In some embodiments, the binding partner includes a sequence at least 70%, at least 75%, at least 80%, at least 85%, at least 90%, at least 95%, at least 99%, or 100% identical to SEQ ID NO: 1, 2, 3, 4, 43, 44, 45, or 46, or a fragment or variant thereof. In some embodiments, the binding partner comprises the sequence of SEQ ID NO: 1 or 43. In some embodiments, the binding partner includes a sequence at least 70%, at least 75%, at least 80%, at least 85%, at least 90%, at least 95%, at least 99%, or 100% identical to SEQ ID NO: 1 or 43. In some embodiments, the binding partner comprises the sequence of SEQ ID NO: 2 or 44. In some embodiments, the binding partner includes a sequence at least 70%, at least 75%, at least 80%, at least 85%, at least 90%, at least 95%, at least 99%, or 100% identical to SEQ ID NO: 2 or 44. In some embodiments, the binding partner comprises the sequence of SEQ ID NO: 3 or 45. In some embodiments, the binding partner includes a sequence at least 70%, at least 75%, at least 80%, at least 85%, at least 90%, at least 95%, at least 99%, or 100% identical to SEQ ID NO: 3 or 45. In some embodiments, the binding partner comprises the sequence of SEQ ID NO: 4 to 46. In some embodiments, the binding partner includes a sequence at least 70%, at least 75%, at least 80%, at least 85%, at least 90%, at least 95%, at least 99%, or 100% identical to SEQ ID NO: 4 or 46.

In some embodiments, the Endo180 polypeptides provided herein are capable of forming a complex with or binding to a C-terminal sequence of a procollagen. In some embodiments the procollagen is a human procollagen. In some embodiments, the C-terminal sequence of a procollagen comprises a C-terminal polypeptide of proα1(I), proα1(II), proα1(III), proα1(V), proα1(XI), proα2(I), proα2(V), proα2(XI), or proα3(XI), or a fragment thereof. In some embodiments, the C-terminal sequence of a procollagen is or comprises a C-terminal polypeptide of proα1(I). In some embodiments, the Endo180 polypeptides provided herein are capable of forming a complex with or binding to a C-terminal sequence of a procollagen as described in Section II. In some embodiments, the Endo180 polypeptides provided herein are capable of forming a complex with or binding to a C-terminal sequence of a procollagen, wherein the sequence includes a C-prodomain of procollagen and a glycine repeat sequence of procollagen. In some embodiments, the Endo180 polypeptide complexes with or binds a C-prodomain of procollagen and a glycine repeat sequence of procollagen or portions thereof.

### 1. Polynucleotides and Vectors

Also provided are polynucleotides (nucleic acid molecules) encoding Endo180 polypeptides described herein, and vectors for genetically engineering cells to express such polypeptides.

In some embodiments, provided are polynucleotides that encode Endo180 polypeptide. In some aspects, the polynucleotide contains a single nucleic acid sequence, such as a nucleic acid sequence encoding a Endo180 polypeptide.

In some embodiments, the polynucleotide encoding the Endo180 polypeptide contains at least one promoter that is operatively linked to control expression of the Endo180 polypeptide or Endo180 fusion proteins. In some embodiments, the polynucleotide contains two, three, or more promoters operatively linked to control expression of the Endo180 polypeptide.

In some embodiments, expression of the Endo180 polypeptides is inducible or conditional. Thus, in some aspects, the polynucleotide encoding the fusion protein(s) contains a conditional promoter, enhancer, or transactivator. In some such aspects, the conditional promoter, enhancer, or transactivator is an inducible promoter, enhancer, or transactivator or a repressible promoter, enhancer, or transactivator.

In some embodiments, the polynucleotide encodes an Endo180 polypeptide sequence set forth by SEQ ID NO: 2 or 44. In some embodiments, the polynucleotide encodes an Endo180 polypeptide sequence at least 70%, at least 75%, at least 80%, at least 85%, at least 90%, at least 95%, at least 99%, or 100% identical to SEQ ID NO: 2 or 44. In some embodiments, the polynucleotide encodes an Endo180 polypeptide sequence set forth by SEQ ID NO: 4 or 46. In some embodiments, the polynucleotide encodes an Endo180 polypeptide sequence at least 70%, at least 75%, at least 80%, at least 85%, at least 90%, at least 95%, at least 99%, or 100% identical to SEQ ID NO: 4 or 46. In some embodiments, the polynucleotide encoding the Endo180 polypeptide includes a signal peptide. In some embodiments, the signal peptide inform the cellular trafficking of the Endo180 polypeptide. In some embodiments, the polynucleotide encodes a signal peptide sequence set forth by SEQ ID NO: 5. In some embodiments, the polynucleotide encodes a signal peptide sequence at least 70%, at least 75%, at least 80%, at least 85%, at least 90%, at least 95%, at least 99%, or 100% identical to SEQ ID NO: 5. In some embodiments, the polynucleotide encodes an Endo180 polypeptide sequence set forth by SEQ ID NO: 1 or 43. In some embodiments, the polynucleotide encodes an Endo180 polypeptide sequence at least 70%, at least 75%, at least 80%, at least 85%, at least 90%, at least 95%, at least 99%, or 100% identical to SEQ ID NO: 1 or 43. In some embodiments, the polynucleotide encodes an Endo180 polypeptide sequence set forth by SEQ ID NO: 3 or 45. In some embodiments, the polynucleotide encodes an Endo180 polypeptide sequence at least 70%, at least 75%, at least 80%, at least 85%, at least 90%, at least 95%, at least 99%, or 100% identical to SEQ ID NO: 3 or 45.

In some embodiments, the polynucleotide encoding the Endo180 polypeptide is introduced into a composition containing cultured cells (e.g., host cells), such as by retroviral transduction, transfection, or transformation. In some embodiments, this can allow for expression (e.g., production) of the Endo180 polypeptide. In some embodiments, the expressed Endo180 polypeptides are purified.

Also provided are vectors or constructs containing nucleic acid molecules as described herein. In some embodiments, the vectors or constructs contain one or more promoters operatively linked to the nucleic acid molecule encoding the Endo180 polypeptide to drive expression thereof.

In some embodiments, the vector is a viral vector. In some embodiments the viral vector is a retroviral vector. In some embodiments, the retroviral vector is a lentiviral vector. In some embodiments, the retroviral vector is a gammaretroviral vector.

In some embodiments, the vector or construct includes a single promoter that drives the expression of one or more nucleic acid molecules of the polynucleotide.

In some embodiments, the vector is comprised in a virus. In some embodiments, the virus is a pseudovirus. In some embodiments, the virus is a viral-like particle. In some embodiments, the vector is comprised in a cell. In some embodiments, the virus or cell in which the vector is comprised contains a recombinant genome.

### B. Support

As described above, in some aspects, the Endo180 polypeptide described herein is present (e.g., coupled, linked, attached, absorbed) on a support. In some embodiments, the support is a solid support or a surface. In some embodiments, the support is or includes a resin, a particle, a bead, a solid substrate, a membrane, or a combination thereof. For example, the support may be bead or a solid resin. In some embodiments, the bead or resin is a stationary phase, for example a chromatography matrix. In some such embodiments, the Endo180 polypeptide is immobilized on the support. In some cases, the Endo180 polypeptide is immobilized to the support via covalent bonds. In some embodiments, the Endo180 polypeptide is immobilized to the support non-covalently.

In some embodiments, the support is a solid support. Any solid support (surface) can be used for the attachment of the Endo180 polypeptide. Non-limiting examples of solid supports on which the Endo180 polypeptide can be attached or absorbed include a magnetic bead, a polymeric bead, a cell culture plate, a microtiter plate, a membrane, plastic substrates, or a hollow fiber. In some embodiments, the Endo180 polypeptide is covalently attached to the solid support. In some embodiments, the Endo180 polypeptide is covalently linked to a CNBr activated resin. In some embodiments, the Endo180 polypeptide is non-covalently attached to the solid support. For example, when an Endo180 fusion protein containing a heterologous polypeptide sequence, such as an Fc sequence, is used, the solid support may have attached or absorbed thereon a molecule, protein, or other agent capable of binding to the heterologous polypeptide sequence. For example, in some embodiments, when using an Endo180 fusion protein containing an Fc sequence, the support may have attached or absorbed thereon protein A capable of binding to the Fc. Similar strategies and arrangements can be used for the affinity tag biotin and avidin or streptavidin.

Non-limiting examples of solid supports include Dynabeads, agarose (e.g., Sepharose®), acrylamide resin, CNBr activated resin, polystyrene-divinylbenzene (PS-DVB), AminoLink resins, crosslinked resins, and chemically functionalized resins.

In some embodiments, the support contains a stationary phase. Thus, in some embodiments, the Endo180 polypeptide is comprised on a stationary phase (also called chromatography matrix). In some such embodiments, the Endo180 polypeptide is attached to the stationary phase. In some cases, the Endo180 polypeptide is attached to the stationary phase via covalent bonds. In some aspects, the Endo180 polypeptide is attached to the stationary phase non-covalently.

Any material may be employed as a chromatography matrix. In general, a suitable chromatography material is essentially innocuous, such as when used in a packed chromatography column under desired conditions. In some embodiments, the stationary phase remains in a predefined location, such as a predefined position, whereas the location of the sample is being altered. Thus, in some embodiments, the stationary phase is the part of a chromatographic system through which a mobile phase flows (either by gravity flow, flow through, or in a batch mode) and where distribution of the components contained in the liquid phase (either dissolved or dispersed) between the phases occurs.

In some embodiments, the chromatography matrix has the form of a solid or semisolid phase, whereas the sample that contains the target proteins to be isolated/separated is a fluid phase. The chromatography matrix can be a particulate material (of any suitable size and shape) or a monolithic chromatography material, including a paper substrate or membrane. Thus, in some aspects, the chromatography can be both column chromatography as well as planar chromatography. In some embodiments, in addition to standard chromatography columns, columns allowing a bidirectional flow such as PhyTipR columns available from PhyNexus, Inc. San Jose, CA, U.S.A. or pipette tips can be used for column based/flow through mode based methods. Thus, in some cases, pipette tips or columns allowing a bidirectional flow are also comprised by chromatography columns useful in the present methods. In some cases, such as where a particulate matrix material is used, the particulate matrix material may, for example, have a mean particle size of about 5 μm to about 200 μm, or from about 5 μm to about 400 μm, or from about 5 μm to about 600 μm. In some aspects, the chromatography matrix may, for example, be or include a polymeric resin or a metal oxide or a metalloid oxide. In some aspects, such as where planar chromatography is used, the matrix material may be any material suitable for planar chromatography, such as conventional cellulose-based or organic polymer based membranes (for example, a paper membrane, a nitrocellulose membrane or a polyvinylidene difluoride (PVDF) membrane) or silica coated glass plates. In some embodiments, the chromatography matrix/stationary phase is a non-magnetic material or non-magnetizable material. In some embodiments, the chromatography matrix is a magnetic material or magnetizable material.

In some embodiments, non-magnetic or non-magnetizable chromatography stationary phases that are suitable in the present methods include derivatized silica or a crosslinked gel. In some aspects, a crosslinked gel may be based on a natural polymer, such as on a polymer class that occurs in nature. For example, a natural polymer on which a chromatography stationary phase may be based is a polysaccharide. In some cases, a respective polysaccharide is generally crosslinked. An example of a polysaccharide matrix includes, but is not limited to, an agarose gel (for example, Superflow™ agarose or a Sepharose® material such as Superflow™ Sepharose® that are commercially available in different bead and pore sizes) or a gel of crosslinked dextran(s). A further illustrative example is a particulate cross-linked agarose matrix, to which dextran is covalently bonded, that is commercially available (in various bead sizes and with various pore sizes) as Sephadex® or Superdex®, both available from GE Healthcare. Another illustrative example of such a chromatography material is Sephacryl® which is also available in different bead and pore sizes from GE Healthcare.

In some embodiments, a crosslinked gel may also be based on a synthetic polymer, such as on a polymer class that does not occur in nature. In some aspects, such a synthetic polymer on which a chromatography stationary phase is based is a polymer that has polar monomer units, and which is therefore in itself polar. Thus, in some cases, such a polar polymer is hydrophilic. Hydrophilic molecules, also termed lipophobic, in some aspects contain moieties that can form dipole-dipole interactions with water molecules. In general, hydrophobic molecules, also termed lipophilic, have a tendency to separate from water.

Illustrative examples of suitable synthetic polymers are polyacrylamide(s), a styrene-divinylbenzene gel and a copolymer of an acrylate and a diol or of an acrylamide and a diol. An illustrative example is a polymethacrylate gel, commercially available as a Fractogel®. A further example is a copolymer of ethylene glycol and methacrylate, commercially available as a Toyopearl®. In some embodiments, a chromatography stationary phase may also include natural and synthetic polymer components, such as a composite matrix or a composite or a co-polymer of a polysaccharide and agarose, e.g. a polyacrylamide/agarose composite, or of a polysaccharide and N,N′-methylenebisacrylamide. An illustrative example of a copolymer of a dextran and N,N′-methylenebisacrylamide is the above-mentioned Sephacryl® series of material. In some embodiments, a derivatized silica may include silica particles that are coupled to a synthetic or to a natural polymer. Examples of such embodiments include, but are not limited to, polysaccharide grafted silica, polyvinylpyrrolidone grafted silica, polyethylene oxide grafted silica, poly(2-hydroxyethylaspartamide) silica and poly(N-isopropylacrylamide) grafted silica.

In some embodiments, the chromatography matrix is a gel filtration matrix. Generally, a gel filtration can be characterized by the property that it is designed to undergo. A gel filtration matrix in some aspects allows the separation of biological entities largely on the basis of their size. In some such aspects, the respective chromatography matrix is typically a particulate porous material as mentioned above. The chromatography matrix may have a certain exclusion limit, which is typically defined in terms of a molecular weight above which molecules are entirely excluded from entering the pores. In some embodiments, the respective molecular weight defining the size exclusion limit may be selected to be below the weight corresponding to the weight of a target cell. In such an embodiment, the target cell is prevented from entering the pores of the size exclusion chromatography matrix. Likewise, a stationary phase may have pores that are of a size that is smaller than the size of a target protein, e.g., trimeric fusion protein.

In some embodiments, a chromatography matrix employed in the present methods may also include magnetically attractable matter such as one or more magnetically attractable particles or a ferrofluid. A respective magnetically attractable particle may comprise a Endo180 polypeptide. In some cases, magnetically attractable particles may contain diamagnetic, ferromagnetic, paramagnetic or superparamagnetic material. In general, superparamagnetic material responds to a magnetic field with an induced magnetic field without a resulting permanent magnetization. Magnetic particles based on iron oxide are for example commercially available as Dynabeads® from Dynal Biotech, as magnetic MicroBeads from Miltenyi Biotec, as magnetic porous glass beads from CPG Inc., as well as from various other sources, such as Roche Applied Science, BIOCLON, BioSource International Inc., micromod, AMBION, Merck, Bangs Laboratories, Polysciences, or Novagen Inc., to name only a few. Magnetic nanoparticles based on superparamagnetic Co and FeCo, as well as ferromagnetic Co nanocrystals have been described, for example by Hutten, A. et al. (J. Biotech. (2004), 112, 47-63).

In some embodiments, provided is an apparatus that contains at least one arrangement of a stationary phase, such as chromatography column. The apparatus may comprise a sample inlet being fluidly connected to the stationary phase of the arrangement and a sample outlet. See, e.g., Section III.

### II. Trimeric Fusion Proteins

It is contemplated that the methods of purification provided herein may be used to purify therapeutic biologics, such as trimeric fusion proteins containing procollagen and domains thereof, e.g., a C-terminal polypeptide sequence of a procollagen.

As described above, many disease targets exist in trimeric forms and/or require trimerization to become active. For example, TNF-related apoptosis-inducing ligand (TRAIL/Apo2L), which is considered a promising cancer therapeutic, mediates activation of the extrinsic apoptosis pathway in a tumor-specific manner by binding to and trimerizing its functional receptors DR4 or DR5. As another example, some enveloped RNA virus antigens responsible for entry into host cells exist in a trimeric form. Methods of generating, preserving, and/or maintaining conformation of trimeric structures may improve the efficacy of therapeutic compositions, such as biologics, in treating disease. For example, trimerization of TRAIL proteins may facilitate the trimerzation of its functional receptors to promote apoptosis. Likewise, a subunit vaccine in which the trimeric form of RNA viral envelop proteins is maintained may allow access to antigenic sites capable of generating an immune response.

In some embodiments, trimerization of proteins, polypeptides, peptides of interest is accomplished by linking the molecule of interest at its C-terminus (C-terminal linkage) to a trimerization domain to promote trimerization of the monomers. In some embodiments, the trimerization stabilizes the membrane proximal aspect of the protein, polypeptide, peptide of interest in a trimeric configuration. In some embodiments, the proteins, polypeptides, peptides of interest are heterologous polypeptide sequences. A heterologous polypeptide sequence may be any heterologous sequence described herein or any polypeptide sequence suitable for trimerization, for example as a therapeutic and/or prophylactic agent such as a vaccine. In some embodiments, the heterologous polypeptide include any polypeptide that is not a trimerization domain or sequence.

In some embodiments, the heterologous polypeptide sequence is derived from a secreted protein or an ecto-domain of a membrane protein. In some embodiments, the heterologous polypeptide sequence is mammalian.

Non-limiting examples of exogenous multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin Foldon (Miroshnikov et al. 1998 Protein Eng 11:329-414), any of which can be linked to a heterologous polypeptide sequence described herein (e.g., by linkage to the C-terminus of a heterologous polypeptide sequence) to promote trimerization of the heterologous polypeptide sequence.

In some embodiments, one or more peptide linkers (such as a gly-ser linker, for example, a 10 amino acid glycine-serine peptide linker) can be used to link the heterologous polypeptide sequence to the multimerization domain. The trimer can include any of the stabilizing mutations provided herein (or combinations thereof) as long as the heterologous polypeptide sequence trimer retains the desired properties.

To be therapeutically feasible, a desired trimerizing protein moiety for biologic drug designs should satisfy the following criteria. Ideally it should be part of a naturally secreted protein, like immunoglobulin Fc, that is also abundant (non-toxic) in the circulation, human in origin (lack of immunogenicity), relatively stable (long half-life) and capable of efficient self-trimerization which is strengthened by inter-chain covalent disulfide bonds so the trimerized heterologous polypeptide sequences are structurally stable.

Collagen is a family of fibrous proteins that are the major components of the extracellular matrix. It is the most abundant protein in mammals, constituting nearly 25% of the total protein in the body. Collagen plays a major structural role in the formation of bone, tendon, skin, cornea, cartilage, blood vessels, and teeth. The fibrillar types of collagen I, II, III, IV, V, and XI are all synthesized as larger trimeric precursors, called procollagens, in which the central uninterrupted triple-helical domain consisting of hundreds of “G-X-Y” repeats (or glycine repeats) is flanked by non-collagenous domains (NC), the N-propeptide and the C-propeptide. Both the C- and N-terminal extensions are processed proteolytically upon secretion of the procollagen, an event that triggers the assembly of the mature protein into collagen fibrils which forms an insoluble cell matrix. BMP-1 is a protease that recognizes a specific peptide sequence of procollagen near the junction between the glycine repeats and the C-prodomain of collagens and is responsible for the removal of the propeptide. The shed trimeric C-propeptide of type I collagen is found in human sera of normal adults at a concentration in the range of 50-300 ng/mL, with children having a much higher level which is indicative of active bone formation. In people with familial high serum concentration of C-propeptide of type I collagen, the level could reach as high as 1-6 μg/mL with no apparent abnormality, suggesting the C-propeptide is not toxic. Structural study of the trimeric C-propeptide of collagen suggested that it is a tri-lobed structure with all three subunits coming together in a junction region near their N-termini to connect to the rest of the procollagen molecule. Such geometry in projecting proteins to be fused in one direction is similar to that of Fc dimer.

Type I, IV, V and XI collagens are mainly assembled into heterotrimeric forms consisting of either two α-1 chains and one α-2 chain (for Type I, IV, V), or three different a chains (for Type XI), which are highly homologous in sequence. The type II and III collagens are both homotrimers of α-1 chain. For type I collagen, the most abundant form of collagen, stable α(I) homotrimer is also formed and is present at variable levels in different tissues. Most of these collagen C-propeptide chains can self-assemble into homotrimers, when over-expressed alone in a cell. Although the N-propeptide domains are synthesized first, molecular assembly into trimeric collagen begins with the in-register association of the C-propeptides. In some embodiments, the C-propeptide complex is stabilized by the formation of interchain disulfide bonds. In some embodiments, the interchain disulfide bond formation facilitates proper chain registration. In some embodiments, the interchain disulfide bonds form as the in-register association of the C-propeptides occurs. The triple helix of the glycine repeats and is then propagated from the associated C-termini to the N-termini in a zipper-like manner. This knowledge has led to the creation of non-natural types of collagen matrix by swapping the C-propeptides of different collagen chains using recombinant DNA technology. Non-collagenous proteins, such as cytokines and growth factors, also have been fused to the N-termini of either pro-collagens or mature collagens to allow new collagen matrix formation, which is intended to allow slow release of the noncollagenous proteins from the cell matrix. However, under both circumstances, the C-propeptides are required to be cleaved before recombinant collagen fibril assembly into an insoluble cell matrix.

Although, other protein trimerization domains, such as those from GCN4 from yeast fibritin from bacteria phage T4 and aspartate transcarbamoylase of Escherichia coli, have been described previously to allow trimerization of heterologous proteins, none of these trimerizing proteins are human in nature, nor are they naturally secreted proteins. As such, any trimeric fusion proteins would have to be made intracellularly, which not only may fold incorrectly for naturally secreted proteins such as soluble receptors, but also make purification of such fusion proteins from thousands of other intracellular proteins difficult. Moreover, the fatal drawback of using such non-human protein trimerization domains (e.g. from yeast, bacteria phage and bacteria) for trimeric biologic drug design is their presumed immunogenicity in the human body, rendering such fusion proteins ineffective shortly after injecting them into the human body.

The use of collagen in a recombinant polypeptide or fusion protein as described herein thus has many advantages, including: (1) collagen is the most abundant protein secreted in the body of a mammal, constituting nearly 25% of the total proteins in the body; (2) the major forms of collagen naturally occur as trimeric helixes, with their globular C-propeptides being responsible for the initiating of trimerization; (3) the trimeric C-propeptide of collagen proteolytically released from the mature collagen is found naturally at sub microgram/mL level in the blood of mammals and is not known to be toxic to the body; (4) the linear triple helical region of collagen can be included as a linker with predicted 2.9 Å spacing per residue, or excluded as part of the fusion protein so the distance between a protein to be trimerized and the C-propeptide of collagen can be precisely adjusted to achieve an optimal biological activity; (5) the recognition site of BMP1 which cleaves the C-propeptide off the pro-collagen can be mutated or deleted to prevent the disruption of a trimeric fusion protein; (6) the C-propeptide domain self-trimerizes via disulfide bonds between the C-propeptide domains in polypeptide chains, and the C-propeptide domain and/or the trimerized C-propeptide domains in a trimeric fusion protein can provide a tag such as a universal affinity tag (for different fusion partners fused to the same C-propeptide domain), which can be used for purification of any secreted fusion proteins created. Thus, different fusion partner polypeptide sequences can be fused to a C-propeptide domain sequences, the different fusion polypeptides can be expressed together (e.g., using the same host cell comprising expression vectors for the different fusion polypeptides), and each fusion polypeptide upon expression can trimerize to form a trimeric fusion protein, thereby generating a mixture comprising different fusion proteins comprising the same C-propeptide domain as a tag. The different fusion proteins can be efficiently purified together using an Endo180 polypeptide or protein disclosed herein. The last advantage described here makes trimeric fusion protein generated as described herein ideal for purification according to the methods provided herein.

In some embodiments, the heterologous polypeptide sequence is linked to a C-terminal propeptide of procollagen to form a fusion protein. In some embodiments, the C-terminal propeptides of the fusion proteins form inter-polypeptide bonds (e.g., disulfide bonds). In some embodiments, the fusion proteins form trimers. In some embodiments, the fusion protein trimers are referred to a trimeric fusion proteins. In some embodiments, the trimeric fusion protein is recombinantly produced.

In some embodiments, the C-terminal propeptide is of human collagen. In some embodiments, the C-terminal propeptide comprises a C-terminal polypeptide of proα1(I), proα1(II), proα1(III), proα1(V), proα1(XI), proα2(I), proα2(V), proα2(XI), or proα3(XI), or a fragment thereof. In some embodiments, the C-terminal propeptide is or comprises a C-terminal polypeptide of proα1(I).

In some embodiments, the C-terminal propeptide is or comprises the amino acid sequence set forth by SEQ ID NO: 18. In some embodiments, the C-terminal propeptide is an amino acid sequence having at least or about 85%, 90%, 92%, 95%, or 97% sequence identity to sequence of SEQ ID NO: 18. In some embodiments, the C-terminal propeptide is or comprises the amino acid sequence set forth by SEQ ID NO: 19. In some embodiments, the C-terminal propeptide is an amino acid sequence having at least or about 85%, 90%, 92%, 95%, or 97% sequence identity to sequence of SEQ ID NO: 19. In some embodiments, the C-terminal propeptide is or is the amino acid sequence set forth by SEQ ID NO: 30. In some embodiments, the C-terminal propeptide exhibits an amino acid sequence having at least or about 85%, 90%, 92%, 95%, or 97% sequence identity to sequence of SEQ ID NO: 20. In some embodiments, the C-terminal propeptide is or comprises the amino acid sequence set forth by SEQ ID NO: 21. In some embodiments, the C-terminal propeptide is an amino acid sequence having at least or about 85%, 90%, 92%, 95%, or 97% sequence identity to sequence of SEQ ID NO: 21.

In some embodiments, the C-terminal propeptide is or comprises the amino acid sequence set forth by SEQ ID NO: 22. In some embodiments, the C-terminal propeptide is an amino acid sequence having at least or about 85%, 90%, 92%, 95%, or 97% sequence identity to sequence of SEQ ID NO: 22. In some embodiments, the C-terminal propeptide is or comprises the amino acid sequence set forth by SEQ ID NO: 23. In some embodiments, the C-terminal propeptide is an amino acid sequence having at least or about 85%, 90%, 92%, 95%, or 97% sequence identity to sequence of SEQ ID NO: 23. In some embodiments, the C-terminal propeptide is or comprises the amino acid sequence set forth by SEQ ID NO: 24. In some embodiments, the C-terminal propeptide is an amino acid sequence having at least or about 85%, 90%, 92%, 95%, or 97% sequence identity to sequence of SEQ ID NO: 24. In some embodiments, the C-terminal propeptide is or comprises the amino acid sequence set forth by SEQ ID NO: 25. In some embodiments, the C-terminal propeptide is an amino acid sequence having at least or about 85%, 90%, 92%, 95%, or 97% sequence identity to sequence of SEQ ID NO: 25.

In some embodiments, the C-terminal propeptide is or comprises the amino acid sequence set forth by SEQ ID NO: 26. In some embodiments, the C-terminal propeptide is an amino acid sequence having at least or about 85%, 90%, 92%, 95%, or 97% sequence identity to sequence of SEQ ID NO: 26. In some embodiments, the C-terminal propeptide is or comprises the amino acid sequence set forth by SEQ ID NO: 27. In some embodiments, the C-terminal propeptide is an amino acid sequence having at least or about 85%, 90%, 92%, 95%, or 97% sequence identity to sequence of SEQ ID NO: 27. In some embodiments, the C-terminal propeptide is or comprises the amino acid sequence set forth by SEQ ID NO: 28. In some embodiments, the C-terminal propeptide is an amino acid sequence having at least or about 85%, 90%, 92%, 95%, or 97% sequence identity to sequence of SEQ ID NO: 28. In some embodiments, the C-terminal propeptide is or comprises the amino acid sequence set forth by SEQ ID NO: 29. In some embodiments, the C-terminal propeptide is an amino acid sequence having at least or about 85%, 90%, 92%, 95%, or 97% sequence identity to sequence of SEQ ID NO: 29.

In some embodiments, the C-terminal propeptide is or comprises the amino acid sequence set forth by SEQ ID NO: 30. In some embodiments, the C-terminal propeptide is an amino acid sequence having at least or about 85%, 90%, 92%, 95%, or 97% sequence identity to sequence of SEQ ID NO: 30. In some embodiments, the C-terminal propeptide is or comprises the amino acid sequence set forth by SEQ ID NO: 31. In some embodiments, the C-terminal propeptide is an amino acid sequence having at least or about 85%, 90%, 92%, 95%, or 97% sequence identity to sequence of SEQ ID NO: 31. In some embodiments, the C-terminal propeptide is or comprises the amino acid sequence set forth by SEQ ID NO: 32. In some embodiments, the C-terminal propeptide is an amino acid sequence having at least or about 85%, 90%, 92%, 95%, or 97% sequence identity to sequence of SEQ ID NO: 32. In some embodiments, the C-terminal propeptide is or comprises the amino acid sequence set forth by SEQ ID NO: 33. In some embodiments, the C-terminal propeptide is an amino acid sequence having at least or about 85%, 90%, 92%, 95%, or 97% sequence identity to sequence of SEQ ID NO: 33.

In some embodiments, the C-terminal propeptide is or comprises the amino acid sequence set forth by SEQ ID NO: 34. In some embodiments, the C-terminal propeptide is an amino acid sequence having at least or about 85%, 90%, 92%, 95%, or 97% sequence identity to sequence of SEQ ID NO: 34. In some embodiments, the C-terminal propeptide is or comprises the amino acid sequence set forth by SEQ ID NO: 35. In some embodiments, the C-terminal propeptide is an amino acid sequence having at least or about 85%, 90%, 92%, 95%, or 97% sequence identity to sequence of SEQ ID NO: 35. In some embodiments, the C-terminal propeptide is or comprises the amino acid sequence set forth by SEQ ID NO: 36. In some embodiments, the C-terminal propeptide is an amino acid sequence having at least or about 85%, 90%, 92%, 95%, or 97% sequence identity to sequence of SEQ ID NO: 36. In some embodiments, the C-terminal propeptide is or comprises the amino acid sequence set forth by SEQ ID NO: 37. In some embodiments, the C-terminal propeptide is an amino acid sequence having at least or about 85%, 90%, 92%, 95%, or 97% sequence identity to sequence of SEQ ID NO: 37.

In any of the preceding embodiments, the C-terminal propeptide can comprise a sequence comprising glycine-X-Y repeats linked to the N-terminus of any of SEQ ID NOs: 18-41, wherein X and Y are independently any amino acid, or an amino acid sequence at least 85%, 90%, 92%, 95%, or 97% identical thereto capable of forming inter-polypeptide disulfide bonds and trimerizing the recombinant polypeptides. In some embodiments, X and Y are independently proline or hydroxyproline.

In some cases where a heterologous polypeptide sequence is linked to the C-terminal propeptide to form the recombinant polypeptide, the fusion proteins form a trimer resulting in a trimeric fusion protein, e.g., a homotrimeric fusion protein.

In some embodiments, trimeric fusion proteins include individual recombinant polypeptides comprising the same heterologous polypeptide sequence. In some embodiments, trimeric fusion proteins include individual fusion proteins each comprising a different heterologous polypeptide sequence from the other fusion proteins. In some embodiments, trimeric fusion proteins include individual fusion proteins wherein one of the individual fusion proteins comprises a heterologous polypeptide sequence different from the other fusion proteins. In some embodiments, trimeric fusion proteins include individual fusion proteins wherein two of the fusion proteins comprise the same heterologous polypeptide sequence, and the heterologous polypeptide sequence is different from the heterologous polypeptide sequence comprised in the remaining fusion protein.

In some embodiments, the heterologous polypeptide sequence is mammalian. Exemplary trimeric fusion proteins are described in U.S. Pat. Nos. 7,268,116, 7,666,837, 7,691,815, 10,618,949, Publication No. US2020/0190181, and Publication No. US2020/0199187, all of which are incorporated by reference in the entirety. In some embodiments, the heterologous polypeptide sequence contains a TNF family protein or fragment thereof. In some embodiments, the heterologous polypeptide sequence contains a TRAIL protein or fragment or variant thereof. In some embodiments, the heterologous polypeptide sequence contains a 4-1-BBL protein or fragment or variant thereof. In some embodiments, the heterologous polypeptide sequence contains an OX40L protein or fragment or variant thereof.

In some embodiments, the heterologous polypeptide sequence is viral. In some embodiments, the heterologous polypeptide sequence is from an RNA virus. In some embodiments, the RNA virus is selected from the group consisting of an HIV, an RSV, a coronavirus, an influenza virus, and a rabies virus. In some embodiments, the heterologous polypeptide contains a coronavirus Spike (S) protein or fragment or variant thereof. In some embodiments, the coronavirus is a SARS-CoV-2. In some embodiments, the coronavirus is a SARS-CoV-1. In some embodiments, the coronavirus is a MERS virus.

In some embodiments, the heterologous polypeptide contains s an HIV gp120 protein or fragment or variant thereof. In some embodiments, the heterologous polypeptide contains an RSV F protein or fragment or variant thereof. In some embodiments, the heterologous polypeptide contains an influenza virus HA protein or fragment or variant thereof. In some embodiments, the heterologous polypeptide comprises a rabies virus G protein or fragment or variant thereof.

In some embodiments, the trimeric fusion proteins described herein are therapeutic, diagnostic, and/or prophylactic agents.

### A. Polynucleotides and Vectors

Also provided are polynucleotides (nucleic acid molecules) encoding the fusion proteins that generate trimeric fusion proteins provided herein, and vectors for genetically engineering cells to express such fusion proteins.

In some embodiments, provided are polynucleotides that encode fusion proteins provided herein. In some embodiments, the fusion proteins are encoded by a recombinant polynucleotide sequence comprising a polynucleotide sequence encoding a heterologous polypeptide sequence fused in-frame to a polynucleotide sequence encoding the C-terminal polypeptide sequence of the procollagen. In some aspects, the polynucleotide contains a single nucleic acid sequence, such as a nucleic acid sequence encoding a fusion protein. In other instances, the polynucleotide contains a first nucleic acid sequence encoding a fusion protein is a particular the heterologous polypeptide sequence and a second nucleic acid sequence encoding a fusion protein comprising a different heterologous polypeptide sequence.

In some embodiments, the polynucleotide encoding the recombinant polypeptide contains at least one promoter that is operatively linked to control expression of the recombinant polypeptide. In some embodiments, the polynucleotide contains two, three, or more promoters operatively linked to control expression of the recombinant polypeptide.

In some embodiments, for example when the polynucleotide contains two or more nucleic acid coding sequences, such as a sequences encoding fusion proteins containing different heterologous polypeptide sequences, and at least one promoter is operatively linked to control expression of the two or more nucleic acid sequences. In some embodiments, the polynucleotide contains two, three, or more promoters operatively linked to control expression of the fusion proteins.

In some embodiments, expression of the fusion protein(s) is inducible or conditional. Thus, in some aspects, the polynucleotide encoding the fusion protein(s) contains a conditional promoter, enhancer, or transactivator. In some such aspects, the conditional promoter, enhancer, or transactivator is an inducible promoter, enhancer, or transactivator or a repressible promoter, enhancer, or transactivator. For example, in some embodiments, an inducible or conditional promoter can be used to restrict expression of the fusion proteins to a specific microenvironment. In some embodiments, expression driven by the inducible or conditional promoter is regulated by exposure to an exogenous agent, such as heat, radiation, or drug.

In cases where the polynucleotide contains more than one nucleic acid sequence encoding a fusion protein, the polynucleotide may further include a nucleic acid sequence encoding a peptide between the one or more nucleic acid sequences. In some cases, the nucleic acid positioned between the nucleic acid sequences encodes a peptide that separates the translation products of the nucleic acid sequences during or after translation. In some embodiments, the peptide contains an internal ribosome entry site (IRES), a self-cleaving peptide, or a peptide that causes ribosome skipping, such as a T2A peptide.

In some embodiments, the polynucleotide encoding the fusion protein(s) is introduced into a composition containing cultured cells (e.g., host cells), such as by retroviral transduction, transfection, or transformation. In some embodiments, the fusion protein, e.g., trimeric fusion protein, is expressed from a mammalian cell. In some embodiments, this can allow for expression (e.g., production) of the fusion protein. In some embodiments, the expressed fusion proteins are purified according to the methods of purification provided herein.

Also provided are vectors or constructs containing nucleic acid molecules as described herein. In some embodiments, the vectors or constructs contain one or more promoters operatively linked to the nucleic acid molecule encoding the fusion protein to drive expression thereof. In some embodiments, the promoter is operatively linked to one or more than one nucleic acid molecule, e.g., nucleic acid molecule encoding fusion proteins containing different heterologous polypeptide sequence.

In some embodiments, the vector is a viral vector. In some embodiments the viral vector is a retroviral vector. In some embodiments, the retroviral vector is a lentiviral vector. In some embodiments, the retroviral vector is a gammaretroviral vector.

In some embodiments, the vector or construct includes a single promoter that drives the expression of one or more nucleic acid molecules of the polynucleotide. In some embodiments, such promoters can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., encoding different recombinant polypeptides) by a message from a single promoter. In some embodiments, the vectors provided herein are bicistronic, allowing the vector to contain and express two nucleic acid sequences. In some embodiments, the vectors provided herein are tricistronic, allowing the vector to contain and express three nucleic acid sequences.

In some embodiments, a single promoter directs expression of an RNA that contains, in a single open reading frame (ORF), two or three genes (e.g. encoding the chimeric signaling receptor and encoding a recombinant receptor) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein include, without limitation, 2A sequences from the foot-and-mouth disease virus (F2A), equine rhinitis A virus (E2A), Thosea asigna virus (T2A), and porcine teschovirus-1 (P2A) as described in U.S. Patent Publication No. 20070116690.

In some embodiments, the vector is comprised in a virus. In some embodiments, the virus is a pseudovirus. In some embodiments, the virus is a viral-like particle. In some embodiments, the vector is comprised in a cell. In some embodiments, the virus or cell in which the vector is comprised contains a recombinant genome.

### III. Articles of Manufacture, Kits, or Apparatus

Also provided are articles of manufacture, kits, or apparatus containing the Endo180 polypeptides and supports as described herein. The articles of manufacture, kits, or apparatus may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include solution bags, vials, including those with stoppers pierceable by a needle for injection. In some embodiments, the articles of manufacture, kits, or apparatus contains resins and columns suitable for purification methods described herein.

The article of manufacture or apparatus may further include a package insert indicating that the compositions can be used for purifying therapeutic proteins or biologics containing a C-terminal polypeptide sequence of a procollagen, such as trimeric fusion proteins described herein. The article of manufacture, kit, or apparatus may further include another or the same container comprising a pharmaceutically-acceptable buffer. It may further include other materials such as other buffers, diluents, filters, needles, and/or syringes.

In some embodiments is an apparatus for purification according to the methods described herein. The apparatus may include a solid support and/or containers being fluidly connected, for example in series.

The apparatus may include a sample inlet being fluidly connected to the solid support contained in a container. The apparatus may also comprise a sample outlet, the sample outlet being fluidly connected to the container containing the solid support.

In some embodiments, the apparatus is functionally closed system. In some embodiments, system is sterile.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. In some embodiments, sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### EXAMPLES

The following examples are included for illustrative purposes only and are not intended to limit the scope of the present disclosure.

### Example 1 Generation of Soluble Endo180 Polypeptides

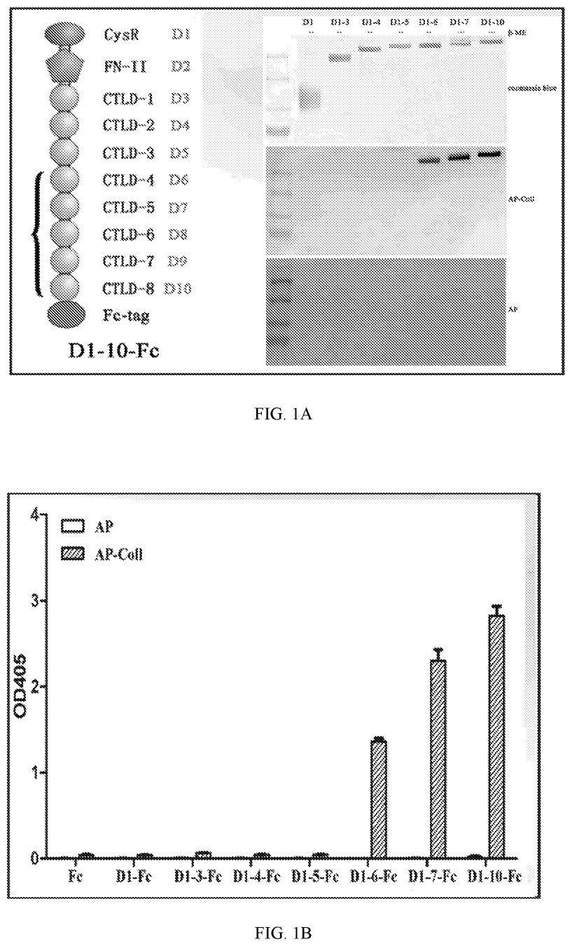

cDNAs encoding serial deletions of human Endo180 domains as indicated in FIG. 1A (domains D1, D1-3, D1-4, D1-5, D1-6, D1-7, or D1-10 retained) were generated by PCR from a full length cDNA template of Endo180 and subcloned into pGH-hFc expression vector (GenHunter Corporation) to allow in-frame fusion to human IgG Fc. The resulting expression vectors each encoding exemplary Endo-180-Fc fusion protein were transfected into GH-CHO cells. Stable clones expressing high levels of each fusion protein were selected via step-wise gene amplifications under increasing MTX concentration. The exemplary recombinant Endo-180-Fc fusion proteins were produced in a Fed-batch serum-free cell culture as secreted proteins and purified to homogeneity by MAbSelectSure Protein A Sepharose resin (GE Biosciences). The resulting exemplary fusion proteins, with Fc alone as a negative control, were separated by a non-reducing SDS-PAGE (FIG. 1A; right upper panel showing Coomassie blue staining).

To determine which Endo180 domain was involved in collagen binding, a human placental alkaline phosphatase and collagen fusion construct (AP-Coll) containing the C-terminal glycine repeat region and prodomain of rat col1a1 (accession number Z78279) was used as an affinity probe after the exemplary Endo180-Fc fusion proteins were transferred to a PVDF membrane. Upon visualization of any bound AP (Alkaline phosphatase) activity, the results showed that collagen binding activity resides in D6 or thereafter of Endo180 (right middle panel, AP-Coll). The AP alone was used a negative control (FIG. 1A; right lower panel).

To further confirm and quantify the Endo180 and collagen binding, each Endo180-Fc fusion protein was captured on a 96-well microtiter plate that was pre-coated with protein A (Prospect). The exemplary Endo180-Fc fusion proteins that were able to bind to AP-Coll were determined by bound AP (Alkaline phosphatase) activity (FIG. 1B), which confirmed the results obtained in FIG. 1A.

### Example 2 Identification of Endo180 Domains for Procollagen Binding

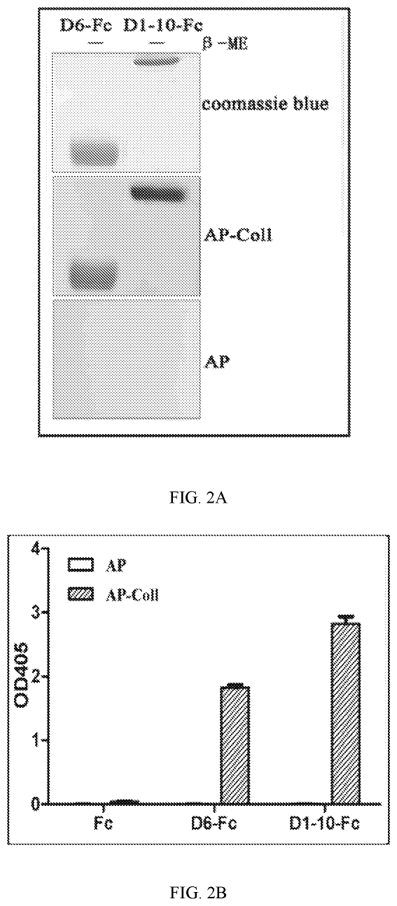

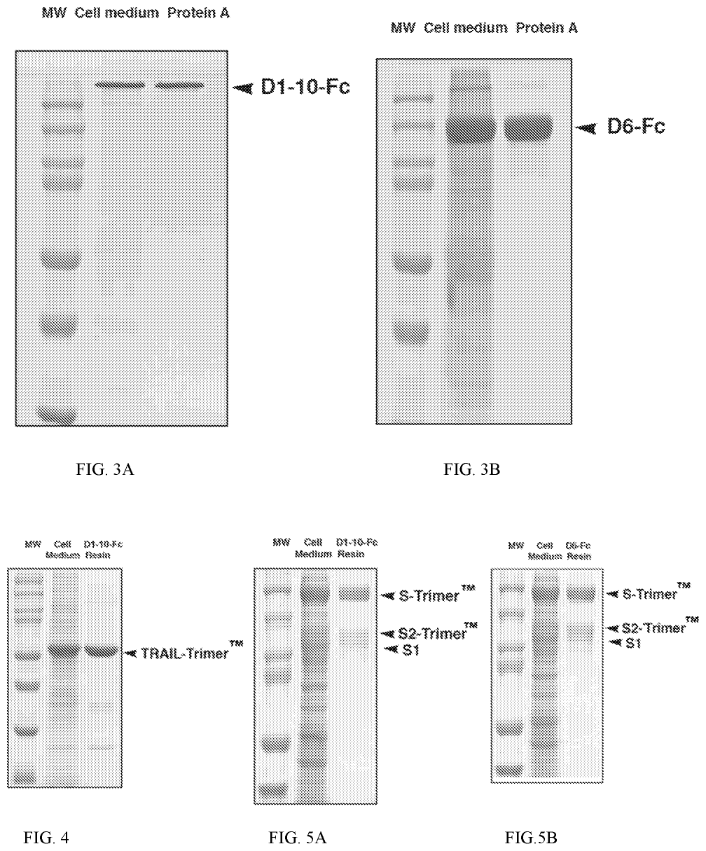

To further dissect which Endo180 domain from D6 on was responsible for procollagen binding, additional Fc fusion proteins were constructed following the same strategy as described in Example 1. As shown in FIG. 2A, domain 6 (D6) alone, which encodes the 4th CTLD, when fused to Fc (D6-Fc), was capable of procollagen binding, when D1-10-Fc served as a positive control.

To further confirm this finding, exemplary D6-Fc and D1-10-Fc fusion proteins were captured on a 96-well microtiter plate that was pre-coated with protein A (Prospect), and bound AP-Coll was determined by AP (Alkaline phosphatase) activity as above (FIG. 2B). The observed AP-Coll binding was supportive of the results observed in FIG. 2A, suggesting that D6 alone was capable of procollagen binding.

### Example 3 Affinity Purification of Trimeric Fusion Proteins Using Endo180-Fc Fusion Proteins as an Affinity Tag Covalently Linked to CNBr Activated Resins

To determine the ability of exemplary Endo180-Fc fusion proteins to act as an affinity tag for purification of trimeric fusion proteins containing collagen, an exemplary Endo180 fusion protein, D1-10-Fc, was covalently immobilized to a CNBr activated resin (Nawei BioSciences). An exemplary human TRAIL-Trimer fusion protein (Liu et al., (2017) Sci Rep. 7(1):8953) was expressed in GH-CHO cells as a secreted protein, and the cell culture medium containing TRAIL-Trimer fusion protein was loaded onto the D1-10-Fc immobilized column. After washing off any unbound contaminating proteins, the bound TRAIL-Trimer™ fusion protein was purified to near homogeneity after elusion with 0.5 M NaCl in phosphate buffered saline (FIG. 4).

### Example 4 Affinity Purification of Trimeric Fusion Proteins Using Endo180-Fc Fusion Proteins as Affinity Tags Non-Covalently Bound to Protein a Sepharose Resins

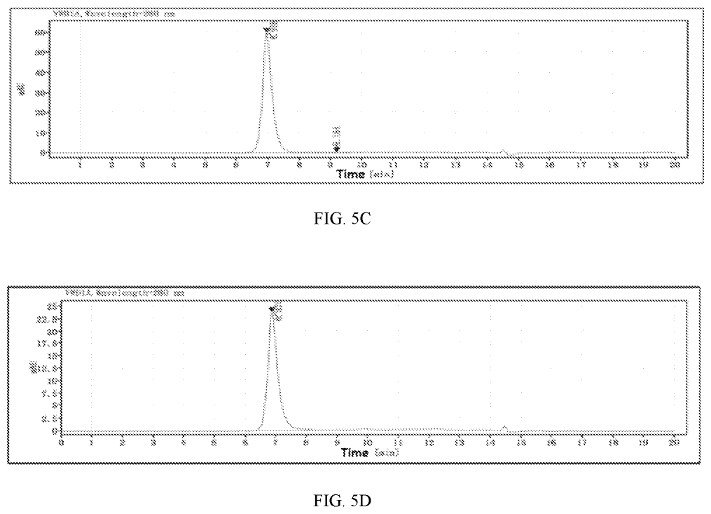

To determine the ability of exemplary D1-10-Fc and D6-Fc Endo180 fusion proteins to act as an affinity tag for purification of trimeric fusion proteins containing collagen, D1-10-Fc or D6-Fc fusion proteins were captured by Protein A Sepharose resins (PresmA, GE BioSciences). An exemplary S-Trimer™ fusion protein (a native SARS-Cov-2 Spike protein antigen fused to collagen Trimer-Tag™) was expressed in GH-CHO cells as a secreted protein, and the cell culture medium containing S-Trimer™ was loaded onto Protein A Sepharose resins with either D1-10-Fc or D6-Fc pre-captured to the column via the high-affinity interaction between Protein A and Fc. After washing off any unbound contaminating proteins, the bound S-Trimer™ was purified to near homogeneity in a single step using 0.5 M-1.0 M NaCl in phosphate buffered saline, respectively as analyzed by SDS-PAGE as indicated in FIG. 5A (D1-10-Fc) and FIG. 5B (D6-Fc).

The purity of the partially cleaved S-Trimer™ at S1/S2 boundary by furin protease purified from either D1-10-Fc or D6-Fc affinity tag was analyzed by Size-exclusion SEC-HPLC (FIGS. 5C and 5D, respectively). The results showed that the partially cleaved S-Trimer™ existed as a single peak, indicating the cleaved S1 eluted from the affinity columns appeared to still be bound to the S2-Trimer™.

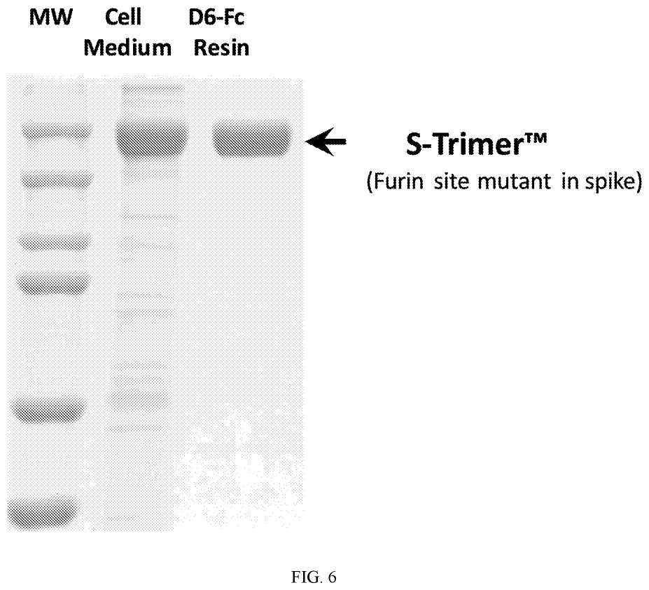

To further determine the ability of exemplary D6-Fc fusion protein to act as an affinity tag for purification of trimeric fusion proteins containing collagen C-prodomain, D6-Fc fusion protein was captured by Protein A Sepharose resins (PresmA, GE BioSciences) and used to purify another form of S-Trimer™ fusion protein (a SARS-Cov-2 Spike protein antigen with furin cleavage site mutation). This exemplary S-Trimer™ fusion protein was expressed in GH-CHO cells as a secreted protein, and the serum-free cell culture medium containing S-Trimer™ was loaded onto Protein A Sepharose resins with D6-Fc (FIG. 6) pre-captured to the column via the high-affinity interaction between Protein A and Fc. After washing off any unbound contaminating proteins, the bound furin site mutant S-Trimer™ was purified to near homogeneity in a single step using 0.5 M-1.0 M NaCl in phosphate buffered saline, as analyzed by SDS-PAGE as indicated. Note, in comparison with that of native S-Trimer™, the purified furin site mutant from of S-Trimer™ did not show any S1 subunit due to furin cleavage (FIG. 6).

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCE LISTING

| SEQ ID | SEQUENCE | ANNOTATION |
|---|---|---|
| 1 | MGPGRPAPAPWPRHLLRCVLLLGCLHLGRPGAPGDAALPEPNVFLIFSHGLQGC | Full length |
| | LEAQGGQVRVTPACNTSLPAQRWKWVSRNRLFNLGTMQCLGTGWPGTNTTASLG | Endo180-Fc (D1- |
| | MYECDREALNLRWHCRTLGDQLSLLLGARTSNISKPGTLERGDQTRSGQWRIYG | 10-Fc) |
| | SEEDLCALPYHEVYTIQGNSHGKPCTIPFKYDNQWFHGCTSTGREDGHLWCATT | |
| | QDYGKDERWGFCPIKSNDCETFWDKDQLTDSCYQFNFQSTLSWREAWASCEQQG | |
| | ADLLSITEIHEQTYINGLLTGYSSTLWIGLNDLDTSGGWQWSDNSPLKYLNWES | |
| | DQPDNPSEENCGVIRTESSGGWQNRDCSIALPYVCKKKPNATAEPTPPDRWANV | |
| | KVECEPSWQPFQGHCYRLQAEKRSWQESKKACLRGGGDLVSIHSMAELEFITKQ | |
| | IKQEVEELWIGLNDLKLQMNFEWSDGSLVSFTHWHPFEPNNERDSLEDCVTIWG | |
| | PEGRWNDSPCNQSLPSICKKAGQLSQGAAEEDHGCRKGWTWHSPSCYWLGEDQV | |
| | TYSEARRLCTDHGSQLVTITNRFEQAFVSSLIYNWEGEYFWTALQDLNSTGSFF | |
| | WLSGDEVMYTHWNRDQPGYSRGGCVALATGSAMGLWEVKNCTSFRARYICRQSL | |
| | GTPVTPELPGPDPTPSLTGSCPQGWASDTKLRYCYKVFSSERLQDKKSWVQAQG | |
| | ACQELGAQLLSLASYEEEHFVANMLNKIFGESEPEIHEQHWFWIGLNRRDPRGG | |
| | QSWRWSDGVGFSYHNFDRSRHDDDDIRGCAVLDLASLQWVAMQCDTQLDWICKI | |
| | PRGTDVREPDDSPQGRREWLRFQEAEYKFFEHHSTWAQAQRICTWFQAELTSVH | |
| | SQAELDFLSHNLQKFSRAQEQHWWIGLHTSESDGRFRWTDGSIINFISWAPGKP | |
| | RPVGKDKKCVYMTASREDWGDQRCLTALPYICKRSNVTKETQPPDLPTTALGGC | |
| | PSDWIQFLNKCFQVQGQEPQSRVKWSEAQFSCEQQEAQLVTITNPLEQAFITAS | |
| | LPNVTFDLWIGLHASQRDFQWVEQEPLMYANWAPGEPSGPSPAPSGNKPTSCAV | |
| | VLHSPSAHFTGRWDDRSCTEETHGFICQKGTDPSLSPSPAALPPAPGTELSYLN | |
| | GTFRLLQKPLRWHDALLLCESRNASLAYVPDPYTQAFLTQAARGLRTPLWIGLA | |
| | GEEGSRRYSWVSEEPLNYVGWQDGEPQQPGGCTYVDVDGAWRTTSCDTKLQGAV | |
| | CGVSSGPPPPRRISYHGSCPQGLADSAWIPFREHCYSFHMELLLGHKEARQRCQ | |
| | RAGGAVLSILDEMENVFVWEHLQSYEGQSRGAWLGMNFNPKGGTLVWQDNTAVN | |
| | YSNWGPPGLGPSMLSHNSCYWIQSNSGLWRPGACTNITMGVVCKQAYVRSEPKS | |
| | CDKTHTCPPCPAPELLGGPSVFLFPPKPKDTLMISRTPEVTCVVVDVSHEDPEV | |
| | KFNWYVDGVEVHNAKTKPREEQYNSTYRVVSVLTVLHQDWLNGKEYKCKVSNKA | |
| | LPAPIEKTISKAKGQPREPQVYTLPPSRDELTKNQVSLTCLVKGFYPSDIAVEW | |
| | ESNGQPENNYKTTPPVLDSDGSFFLYSKLTVDKSRWQQGNVFSCSVMHEALHNH | |
| | YTQKSLSLSPGK | |
| 2 | GAPGDAALPEPNVFLIFSHGLQGCLEAQGGQVRVTPACNTSLPAQRWKWVSRNR | Mature Endo-180- |
| | LFNLGTMQCLGTGWPGTNTTASLGMYECDREALNLRWHCRTLGDQLSLLLGART | Fc (D1-10-Fc) |
| | SNISKPGTLERGDQTRSGQWRIYGSEEDLCALPYHEVYTIQGNSHGKPCTIPFK | |
| | YDNQWFHGCTSTGREDGHLWCATTQDYGKDERWGFCPIKSNDCETFWDKDQLTD | |
| | SCYQFNFQSTLSWREAWASCEQQGADLLSITEIHEQTYINGLLTGYSSTLWIGL | |
| | NDLDTSGGWQWSDNSPLKYLNWESDQPDNPSEENCGVIRTESSGGWQNRDCSIA | |
| | LPYVCKKKPNATAEPTPPDRWANVKVECEPSWQPFQGHCYRLQAEKRSWQESKK | |
| | ACLRGGGDLVSIHSMAELEFITKQIKQEVEELWIGLNDLKLQMNFEWSDGSLVS | |
| | FTHWHPFEPNNFRDSLEDCVTIWGPEGRWNDSPCNQSLPSICKKAGQLSQGAAE | |
| | EDHGCRKGWTWHSPSCYWLGEDQVTYSEARRLCTDHGSQLVTITNRFEQAFVSS | |
| | LIYNWEGEYFWTALQDLNSTGSFFWLSGDEVMYTHWNRDQPGYSRGGCVALATG | |
| | SAMGLWEVKNCTSFRARYICRQSLGTPVTPELPGPDPTPSLTGSCPQGWASDTK | |
| | LRYCYKVFSSERLQDKKSWVQAQGACQELGAQLLSLASYEEEHFVANMLNKIFG | |
| | ESEPEIHEQHWFWIGLNRRDPRGGQSWRWSDGVGFSYHNFDRSRHDDDDIRGCA | |
| | VLDLASLQWVAMQCDTQLDWICKIPRGTDVREPDDSPQGRREWLRFQEAEYKFF | |
| | EHHSTWAQAQRICTWFQAELTSVHSQAELDFLSHNLQKFSRAQEQHWWIGLHTS | |
| | ESDGRFRWTDGSIINFISWAPGKPRPVGKDKKCVYMTASREDWGDQRCLTALPY | |
| | ICKRSNVTKETQPPDLPTTALGGCPSDWIQFLNKCFQVQGQEPQSRVKWSEAQF | |
| | SCEQQEAQLVTITNPLEQAFITASLPNVTFDLWIGLHASQRDFQWVEQEPLMYA | |
| | NWAPGEPSGPSPAPSGNKPTSCAVVLHSPSAHFTGRWDDRSCTEETHGFICQKG | |
| | TDPSLSPSPAALPPAPGTELSYLNGTFRLLQKPLRWHDALLLCESRNASLAYVP | |
| | DPYTQAFLTQAARGLRTPLWIGLAGEEGSRRYSWVSEEPLNYVGWQDGEPQQPG | |
| | GCTYVDVDGAWRTTSCDTKLQGAVCGVSSGPPPPRRISYHGSCPQGLADSAWIP | |
| | FREHCYSFHMELLLGHKEARQRCQRAGGAVLSILDEMENVFVWEHLQSYEGQSR | |
| | GAWLGMNFNPKGGTLVWQDNTAVNYSNWGPPGLGPSMLSHNSCYWIQSNSGLWR | |
| | PGACTNITMGVVCKQAYVRSEPKSCDKTHTCPPCPAPELLGGPSVFLFPPKPKD | |
| | TLMISRTPEVTCVVVDVSHEDPEVKFNWYVDGVEVHNAKTKPREEQYNSTYRVV | |
| | SVLTVLHQDWLNGKEYKCKVSNKALPAPIEKTISKAKGQPREPQVYTLPPSRDE | |
| | LTKNQVSLTCLVKGFYPSDIAVEWESNGQPENNYKTTPPVLDSDGSFFLYSKLT | |
| | VDKSRWQQGNVFSCSVMHEALHNHYTQKSLSLSPGK | |
| 3 | MGPGRPAPAPWPRHLLRCVLLLGCLHLGRPRSPVTPELPGPDPTPSLTGSCPQG | Full Length D6- |
| | WASDTKLRYCYKVFSSERLQDKKSWVQAQGACQELGAQLLSLASYEEEHFVANM | Fc |
| | LNKIFGESEPEIHEQHWFWIGLNRRDPRGGQSWRWSDGVGFSYHNFDRSRHDDD | |
| | DIRGCAVLDLASLQWVAMQCDTQLDWICKIPRSEPKSCDKTHTCPPCPAPELLG | |
| | GPSVFLFPPKPKDTLMISRTPEVTCVVVDVSHEDPEVKENWYVDGVEVHNAKTK | |
| | PREEQYNSTYRVVSVLTVLHQDWLNGKEYKCKVSNKALPAPIEKTISKAKGQPR | |
| | EPQVYTLPPSRDELTKNQVSLTCLVKGFYPSDIAVEWESNGQPENNYKTTPPVL | |
| | DSDGSFFLYSKLTVDKSRWQQGNVFSCSVMHEALHNHYTQKSLSLSPGK | |
| 4 | RSPVTPELPGPDPTPSLTGSCPQGWASDTKLRYCYKVFSSERLQDKKSWVQAQG | Mature D6-Fc |
| | ACQELGAQLLSLASYEEEHFVANMLNKIFGESEPEIHEQHWFWIGLNRRDPRGG | |
| | QSWRWSDGVGFSYHNFDRSRHDDDDIRGCAVLDLASLQWVAMQCDTQLDWICKI | |
| | PRSEPKSCDKTHTCPPCPAPELLGGPSVFLFPPKPKDTLMISRTPEVTCVVVDV | |
| | SHEDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSVM | |
| | HEALHNHYTQKSLSLSPGK | |
| 5 | MGPGRPAPAPWPRHLLRCVLLLGCLHLGRP | Signal Peptide |
| 6 | EPKSCDKTHTCPPCPAPELLGGPSVFLFPPKPKDTLMISRTPEVTCVVVDVSHE | Fc |
| | DPEVKFNWYVDGVEVHNAKTKPREEQYNSTYRVVSVLTVLHQDWLNGKEYKCKV | |
| | SNKALPAPIEKTISKAKGQPREPQVYTLPPSRDELTKNQVSLTCLVKGFYPSDI | |
| | AVEWESNGQPENNYKTTPPVLDSDGSFFLYSKLTVDKSRWQQGNVFSCSVMHEA | |
| | LHNHYTQKSLSLSPGK | |
| 7 | GAPGDAALPEPNVFLIFSHGLQGCLEAQGGQVRVTPACNTSLPAQRWKWVSRNR | Mature Endo180 |
| | LFNLGTMQCLGTGWPGTNTTASLGMYECDREALNLRWHCRTLGDQLSLLLGART | Ecto-domain |
| | SNISKPGTLERGDQTRSGQWRIYGSEEDLCALPYHEVYTIQGNSHGKPCTIPFK | (Uniprot Q9UBG0) |
| | YDNQWFHGCTSTGREDGHLWCATTQDYGKDERWGFCPIKSNDCETFWDKDQLTD | |
| | SCYQFNFQSTLSWREAWASCEQQGADLLSITEIHEQTYINGLLTGYSSTLWIGL | |
| | NDLDTSGGWQWSDNSPLKYLNWESDQPDNPSEENCGVIRTESSGGWQNRDCSIA | |
| | LPYVCKKKPNATAEPTPPDRWANVKVECEPSWQPFQGHCYRLQAEKRSWQESKK | |
| | ACLRGGGDLVSIHSMAELEFITKQIKQEVEELWIGLNDLKLQMNFEWSDGSLVS | |
| | FTHWHPFEPNNERDSLEDCVTIWGPEGRWNDSPCNQSLPSICKKAGQLSQGAAE | |
| | EDHGCRKGWTWHSPSCYWLGEDQVTYSEARRLCTDHGSQLVTITNRFEQAFVSS | |
| | LIYNWEGEYFWTALQDLNSTGSFFWLSGDEVMYTHWNRDQPGYSRGGCVALATG | |
| | SAMGLWEVKNCTSFRARYICRQSLGTPVTPELPGPDPTPSLTGSCPQGWASDTK | |
| | LRYCYKVFSSERLQDKKSWVQAQGACQELGAQLLSLASYEEEHFVANMLNKIFG | |
| | ESEPEIHEQHWFWIGLNRRDPRGGQSWRWSDGVGFSYHNFDRSRHDDDDIRGCA | |
| | VLDLASLQWVAMQCDTQLDWICKIPRGTDVREPDDSPQGRREWLRFQEAEYKFF | |
| | EHHSTWAQAQRICTWFQAELTSVHSQAELDFLSHNLQKFSRAQEQHWWIGHTS | |
| | ESDGRFRWTDGSIINFISWAPGKPRPVGKDKKCVYMTASREDWGDQRCLTALPY | |
| | ICKRSNVTKETQPPDLPTTALGGCPSDWIQFLNKCFQVQGQEPQSRVKWSEAQF | |
| | SCEQQEAQLVTITNPLEQAFITASLPNVTFDLWIGLHASQRDFQWVEQEPLMYA | |
| | NWAPGEPSGPSPAPSGNKPTSCAVVLHSPSAHFTGRWDDRSCTEETHGFICQKG | |
| | TDPSLSPSPAALPPAPGTELSYLNGTFRLLQKPLRWHDALLLCESRNASLAYVP | |
| | DPYTQAFLTQAARGLRTPLWIGLAGEEGSRRYSWVSEEPLNYVGWQDGEPQQPG | |
| | GCTYVDVDGAWRTTSCDTKLQGAVCGVSSGPPPPRRISYHGSCPQGLADSAWIP | |
| | FREHCYSFHMELLLGHKEARQRCQRAGGAVLSILDEMENVFVWEHLQSYEGQSR | |
| | GAWLGMNFNPKGGTLVWQDNTAVNYSNWGPPGLGPSMLSHNSCYWIQSNSGLWR | |
| | PGACTNITMGVVCKLPRAEQSSFSPSALPENPAA | |
| 8 | PNVFLIFSHGLQGCLEAQGGQVRVTPACNTSLPAQRWKWVSRNRLENLGTMQCL | D1 (Ricin B-type |
| | GTGWPGTNTTASLGMYECDREALNLRWHCRTLGDQLSLLLGARTSNISKPGTLE | lectin domain) |
| | RGDQTRSGQWRIYGSEEDL | |
| 9 | SHGKPCTIPFKYDNQWFHGCTSTGREDGHLWCATTQDYGKDERWGFCPI | D2 (Fibronectin |
| | | type-II domain) |
| 10 | LTDSCYQFNFQSTLSWREAWASCEQQGADLLSITEIHEQTYINGLLTGYSSTLW | D3 (C-type |
| | IGLNDLDTSGGWQWSDNSPLKYLNWESDQPDNPSEENCGVIRTESSGGWQNRDC | lectin 1) |
| | SIALPYVCK | |
| 11 | FQGHCYRLQAEKRSWQESKKACLRGGGDLVSIHSMAELEFITKQIKQEVEELWI | D4 (C-type |
| | GLNDLKLQMNFEWSDGSLVSFTHWHPFEPNNERDSLEDCVTIWGPEGRWNDSPC | lectin 2) |
| | NQSLPSICK | |
| 12 | HSPSCYWLGEDQVTYSEARRLCTDHGSQLVTITNRFEQAFVSSLIYNWEGEYFW | D5 (C-type |
| | TALQDLNSTGSFFWLSGDEVMYTHWNRDQPGYSRGGCVALATGSAMGLWEVKNC | lectin 3) |
| | TSFRARYIC | |
| 13 | KLRYCYKVFSSERLQDKKSWVQAQGACQELGAQLLSLASYEEEHFVANMLNKIF | D6 (C-type |
| | GESEPEIHEQHWFWIGLNRRDPRGGQSWRWSDGVGFSYHNFDRSRHDDDDIRGC | lectin 4) |
| | AVLDLASLQWVAMQCDTQLDWICK | |
| 14 | FQEAEYKFFEHHSTWAQAQRICTWFQAELTSVHSQAELDFLSHNLQKESRAQEQ | D7 (C-type |
| | HWWIGLHTSESDGRFRWTDGSIINFISWAPGKPRPVGKDKKCVYMTASREDWGD | lectin 5) |
| | QRCLTALPYICK | |
| 15 | FLNKCFQVQGQEPQSRVKWSEAQFSCEQQEAQLVTITNPLEQAFITASLPNVTF | D8 (C-type |
| | DLWIGLHASQRDFQWVEQEPLMYANWAPGEPSGPSPAPSGNKPTSCAVVLHSPS | lectin 6) |
| | AHFTGRWDDRSCTEETHGFIC | |
| 16 | YLNGTFRLLQKPLRWHDALLLCESRNASLAYVPDPYTQAFLTQAARGLRTPLWI | D9 (C-type |
| | GLAGEEGSRRYSWVSEEPLNYVGWQDGEPQQPGGCTYVDVDGAWRTTSCDTKLQ | lectin 7) |
| | GAVC | |
| 17 | FREHCYSFHMELLLGHKEARQRCQRAGGAVLSILDEMENVFVWEHLQSYEGQSR | D10 (C-type |
| | GAWLGMNFNPKGGTLVWQDNTAVNYSNWGPPGLGPSMLSHNSCYWIQSNSGLWR | lectin 8) |
| | PGACTNITMGVVC | |
| 18 | NGLPGPIGPPGPRGRTGDAGPVGPPGPPGPPGPPGPPSAGFDFSFLPQPPQEKA | Trimerization |
| | HDGGRYYRANDANVVRDRDLEVDTTLKSLSQQIENIRSPEGSRKNPARTCRDLK | peptide (Type I) |
| | MCHSDWKSGEYWIDPNQGCNLDAIKVFCNMETGETCVYPTQPSVAQKNWYISKN | with glycine-X-Y |
| | PKDKRHVWFGESMTDGFQFEYGGQGSDPADVAIQLTFLRLMSTEASQNITYHCK | repeats and D→N |
| | NSVAYMDQQTGNLKKALLLKGSNEIEIRAEGNSRFTYSVTVDGCTSHTGAWGKT | mutation at BMP- |
| | VIEYKTTKSSRLPIIDVAPLDVGAPDQEFGFDVGPVCFL | 1 site, KS |
| | | version |
| 19 | NGLPGPIGPPGPRGRTGDAGPVGPPGPPGPPGPPGPPSAGFDFSFLPQPPQEKA | Trimerization |
| | HDGGRYYRNDDANVVRDRDLEVDTTLKSLSQQIENIRSPEGSRKNPARTCRDLK | peptide (Type I) |
| | MCHSDWKSGEYWIDPNQGCNLDAIKVFCNMETGETCVYPTQPSVAQKNWYISKN | with glycine-X-Y |
| | PKDKRHVWFGESMTDGFQFEYGGQGSDPADVAIQLTFLRLMSTEASQNITYHCK | repeats and A→N |
| | NSVAYMDQQTGNLKKALLLKGSNEIEIRAEGNSRFTYSVTVDGCTSHTGAWGKT | mutation at BMP- |
| | VIEYKTTKSSRLPIIDVAPLDVGAPDQEFGFDVGPVCFL | 1 site, KS |
| | | version |
| 20 | RSNGLPGPIGPPGPRGRTGDAGPVGPPGPPGPPGPPGPPSAGFDFSFLPQPPQE | Trimerization |
| | KAHDGGRYYRANDANVVRDRDLEVDTTLKSLSQQIENIRSPEGSRKNPARTCRD | peptide (Type I) |
| | LKMCHSDWKSGEYWIDPNQGCNLDAIKVFCNMETGETCVYPTQPSVAQKNWYIS | with glycine-X-Y |
| | KNPKDKRHVWFGESMTDGFQFEYGGQGSDPADVAIQLTFLRLMSTEASQNITYH | repeats and D→N |
| | CKNSVAYMDQQTGNLKKALLLKGSNEIEIRAEGNSRFTYSVTVDGCTSHTGAWG | mutation at BMP- |
| | KTVIEYKTTKSSRLPIIDVAPLDVGAPDQEFGFDVGPVCFL | 1 site, KS |
| | | version |
| 21 | GSNGLPGPIGPPGPRGRTGDAGPVGPPGPPGPPGPPGPPSAGFDFSFLPQPPQE | Trimerization |
| | KAHDGGRYYRANDANVVRDRDLEVDTTLKSLSQQIENIRSPEGSRKNPARTCRD | peptide (Type I) |
| | LKMCHSDWKSGEYWIDPNQGCNLDAIKVFCNMETGETCVYPTQPSVAQKNWYIS | with glycine-X-Y |
| | KNPKDKRHVWFGESMTDGFQFEYGGQGSDPADVAIQLTFLRLMSTEASQNITYH | repeats and D→N |
| | CKNSVAYMDQQTGNLKKALLLKGSNEIEIRAEGNSRFTYSVTVDGCTSHTGAWG | mutation at BMP- |
| | KTVIEYKTTKSSRLPIIDVAPLDVGAPDQEFGFDVGPVCFL | 1 site, KS |
| | | version |
| 22 | NGLPGPIGPPGPRGRTGDAGPVGPPGPPGPPGPPGPPSAGFDFSFLPQPPQEKA | Trimerization |
| | HDGGRYYRADDANVVRDRDLEVDTTLKSLSQQIENIRSPEGSRKNPARTCRDLK | peptide (Type I) |
| | MCHSDWKSGEYWIDPNQGCNLDAIKVFCNMETGETCVYPTQPSVAQKNWYISKN | with glycine-X-Y |
| | PKDKRHVWFGESMTDGFQFEYGGQGSDPADVAIQLTFLRLMSTEASQNITYHCK | repeats, no BMP- |
| | NSVAYMDQQTGNLKKALLLKGSNEIEIRAEGNSRFTYSVTVDGCTSHTGAWGKT | 1 site mutation, |
| | VIEYKTTKSSRLPIIDVAPLDVGAPDQEFGFDVGPVCFL | KS version |
| 23 | SNGLPGPIGPPGPRGRTGDAGPVGPPGPPGPPGPPGPPSAGFDFSFLPQPPQEK | Trimerization |
| | AHDGGRYYRADDANVVRDRDLEVDTTLKSLSQQIENIRSPEGSRKNPARTCRDL | peptide (Type I) |
| | KMCHSDWKSGEYWIDPNQGCNLDAIKVFCNMETGETCVYPTQPSVAQKNWYISK | with glycine-X-Y |
| | NPKDKRHVWFGESMTDGFQFEYGGQGSDPADVAIQLTFLRLMSTEASQNITYHC | repeats, no BMP- |
| | KNSVAYMDQQTGNLKKALLLKGSNEIEIRAEGNSRFTYSVTVDGCTSHTGAWGK | 1 site mutation, |
| | TVIEYKTTKSSRLPIIDVAPLDVGAPDQEFGFDVGPVCFL | KS version |
| 24 | NGLPGPIGPPGPRGRTGDAGPVGPPGPPGPPGPPGPPSAGFDFSFLPQPPQEKA | Trimerization |
| | HDGGRYYRADDANVVRDRDLEVDTTLKSLSQQIENIRSPEGSRKNPARTCRDLK | peptide (Type I) |
| | MCHSDWKSGEYWIDPNQGCNLDAIKVFCNMETGETCVYPTQPSVAQKNWYISKN | with glycine-X-Y |
| | PKDKRHVWFGESMTDGFQFEYGGQGSDPADVAIQLTFLRLMSTEASQNITYHCK | repeats, no BMP- |
| | NSVAYMDQQTGNLKKALLLQGSNEIEIRAEGNSRFTYSVTVDGCTSHTGAWGKT | 1 site mutation, |
| | VIEYKTTKTSRLPIIDVAPLDVGAPDQEFGFDVGPVCFL | QT version |
| 25 | SNGLPGPIGPPGPRGRTGDAGPVGPPGPPGPPGPPGPPSAGFDFSFLPQPPQEK | Trimerization |
| | AHDGGRYYRADDANVVRDRDLEVDTTLKSLSQQIENIRSPEGSRKNPARTCRDL | peptide (Type I) |
| | KMCHSDWKSGEYWIDPNQGCNLDAIKVFCNMETGETCVYPTQPSVAQKNWYISK | with glycine-X-Y |
| | NPKDKRHVWFGESMTDGFQFEYGGQGSDPADVAIQLTFLRLMSTEASQNITYHC | repeats, no BMP- |
| | KNSVAYMDQQTGNLKKALLLQGSNEIEIRAEGNSRFTYSVTVDGCTSHTGAWGK | 1 site mutation, |
| | TVIEYKTTKTSRLPIIDVAPLDVGAPDQEFGFDVGPVCFL | QT version |
| 26 | ANVVRDRDLEVDTTLKSLSQQIENIRSPEGSRKNPARTCRDLKMCHSDWKSGEY | Trimerization |
| | WIDPNQGCNLDAIKVFCNMETGETCVYPTQPSVAQKNWYISKNPKDKRHVWFGE | peptide (Type |
| | SMTDGFQFEYGGQGSDPADVAIQLTFLRLMSTEASQNITYHCKNSVAYMDQQTG | I), KS version |
| | NLKKALLLKGSNEIEIRAEGNSRFTYSVTVDGCTSHTGAWGKTVIEYKTTKSSR | |
| | LPIIDVAPLDVGAPDQEFGFDVGPVCFL | |
| 27 | DANVVRDRDLEVDTTLKSLSQQIENIRSPEGSRKNPARTCRDLKMCHSDWKSGE | Trimerization |
| | YWIDPNQGCNLDAIKVFCNMETGETCVYPTQPSVAQKNWYISKNPKDKRHVWFG | peptide (Type |
| | ESMTDGFQFEYGGQGSDPADVAIQLTFLRLMSTEASQNITYHCKNSVAYMDQQT | I), KS version |
| | GNLKKALLLKGSNEIEIRAEGNSRFTYSVTVDGCTSHTGAWGKTVIEYKTTKSS | |
| | RLPIIDVAPLDVGAPDQEFGFDVGPVCF | |
| 28 | SDANVVRDRDLEVDTTLKSLSQQIENIRSPEGSRKNPARTCRDLKMCHSDWKSG | Trimerization |
| | EYWIDPNQGCNLDAIKVFCNMETGETCVYPTQPSVAQKNWYISKNPKDKRHVWF | peptide (Type |
| | GESMTDGFQFEYGGQGSDPADVAIQLTFLRLMSTEASQNITYHCKNSVAYMDQQ | I), KS version |
| | TGNLKKALLLKGSNEIEIRAEGNSRFTYSVTVDGCTSHTGAWGKTVIEYKTTKS | |
| | SRLPIIDVAPLDVGAPDQEFGFDVGPVCFL | |
| 29 | RSDANVVRDRDLEVDTTLKSLSQQIENIRSPEGSRKNPARTCRDLKMCHSDWKS | Trimerization |
| | GEYWIDPNQGCNLDAIKVFCNMETGETCVYPTQPSVAQKNWYISKNPKDKRHVW | peptide (Type |
| | FGESMTDGFQFEYGGQGSDPADVAIQLTFLRLMSTEASQNITYHCKNSVAYMDQ | I), KS version |
| | QTGNLKKALLLKGSNEIEIRAEGNSRFTYSVTVDGCTSHTGAWGKTVIEYKTTK | |
| | SSRLPIIDVAPLDVGAPDQEFGFDVGPVCFL | |
| 30 | NGLPGPIGPPGPRGRTGDAGPVGPPGPPGPPGPPGPPSAGFDFSFLPQPPQEKA | Trimerization |
| | HDGGRYYRANDANVVRDRDLEVDTTLKSLSQQIENIRSPEGSRKNPARTCRDLK | peptide (Type |
| | MCHSDWKSGEYWIDPNQGCNLDAIKVFCNMETGETCVYPTQPSVAQKNWYISKN | I), with |
| | PKDKRHVWFGESMTDGFQFEYGGQGSDPADVAIQLTFLRLMSTEASQNITYHCK | glycine-X-Y |
| | NSVAYMDQQTGNLKKALLLQGSNEIEIRAEGNSRFTYSVTVDGCTSHTGAWGKT | repeats and D→N |
| | VIEYKTTKTSRLPIIDVAPLDVGAPDQEFGFDVGPVCFL | mutation at BMP- |
| | | 1 site, QT |
| | | version |
| 31 | NGLPGPIGPPGPRGRTGDAGPVGPPGPPGPPGPPGPPSAGFDFSFLPQPPQEKA | Trimerization |
| | HDGGRYYRNDDANVVRDRDLEVDTTLKSLSQQIENIRSPEGSRKNPARTCRDLK | peptide (Type |
| | MCHSDWKSGEYWIDPNQGCNLDAIKVFCNMETGETCVYPTQPSVAQKNWYISKN | I), with |
| | PKDKRHVWFGESMTDGFQFEYGGQGSDPADVAIQLTFLRLMSTEASQNITYHCK | glycine-X-Y |
| | NSVAYMDQQTGNLKKALLLQGSNEIEIRAEGNSRFTYSVTVDGCTSHTGAWGKT | repeats and A→N |
| | VIEYKTTKTSRLPIIDVAPLDVGAPDQEFGFDVGPVCFL | mutation at BMP- |
| | | 1 site, QT |
| | | version |
| 32 | RSNGLPGPIGPPGPRGRTGDAGPVGPPGPPGPPGPPGPPSAGFDFSFLPQPPQE | Trimerization |
| | KAHDGGRYYRANDANVVRDRDLEVDTTLKSLSQQIENIRSPEGSRKNPARTCRD | peptide (Type |
| | LKMCHSDWKSGEYWIDPNQGCNLDAIKVFCNMETGETCVYPTQPSVAQKNWYIS | I), with |
| | KNPKDKRHVWFGESMTDGFQFEYGGQGSDPADVAIQLTFLRLMSTEASQNITYH | glycine-X-Y |
| | CKNSVAYMDQQTGNLKKALLLQGSNEIEIRAEGNSRFTYSVTVDGCTSHTGAWG | repeats and D→N |
| | KTVIEYKTTKTSRLPIIDVAPLDVGAPDQEFGFDVGPVCFL | mutation at BMP- |
| | | 1 site, QT |
| | | version |
| 33 | GSNGLPGPIGPPGPRGRTGDAGPVGPPGPPGPPGPPGPPSAGFDFSFLPQPPQE | Trimerization |
| | KAHDGGRYYRANDANVVRDRDLEVDTTLKSLSQQIENIRSPEGSRKNPARTCRD | peptide (Type |
| | LKMCHSDWKSGEYWIDPNQGCNLDAIKVFCNMETGETCVYPTQPSVAQKNWYIS | I), with |
| | KNPKDKRHVWFGESMTDGFQFEYGGQGSDPADVAIQLTFLRLMSTEASQNITYH | glycine-X-Y |
| | CKNSVAYMDQQTGNLKKALLLQGSNEIEIRAEGNSRFTYSVTVDGCTSHTGAWG | repeats and D→N |
| | KTVIEYKTTKTSRLPIIDVAPLDVGAPDQEFGFDVGPVCFL | mutation at BMP- |
| | | 1 site, QT |
| | | version |
| 34 | DEIMTSLKSVNGQIESLISPDGSRKNPARNCRDLKFCHPELKSGEYWVDPNQGC | Trimerization |
| | KLDAIKVFCNMETGETCISANPLNVPRKHWWTDSSAEKKHVWFGESMDGGFQFS | peptide (Type |
| | YGNPELPEDVLDVQLAFLRLLSSRASQNITYHCKNSIAYMDQASGNVKKALKLM | III) |
| | GSNEGEFKAEGNSKFTYTVLEDGCTKHTGEWSKTVFEYRTRKAVRLPIVDIAPY | |
| | DIGGPDQEFGVDVGPVCF | |
| 35 | EPMDFKINTDEIMTSLKSVNGQIESLISPDGSRKNPARNCRDLKFCHPELKSGE | Trimerization |
| | YWVDPNQGCKLDAIKVFCNMETGETCISANPLNVPRKHWWTDSSAEKKHVWEGE | peptide (Type |
| | SMDGGFQFSYGNPELPEDVLDVQLAFLRLLSSRASQNITYHCKNSIAYMDQASG | III) |
| | NVKKALKLMGSNEGEFKAEGNSKFTYTVLEDGCTKHTGEWSKTVFEYRTRKAVR | |
| | LPIVDIAPYDIGGPDQEFGVDVGPVCFL | |
| 36 | SEPMDFKINTDEIMTSLKSVNGQIESLISPDGSRKNPARNCRDLKFCHPELKSG | Trimerization |
| | EYWVDPNQGCKLDAIKVFCNMETGETCISANPLNVPRKHWWTDSSAEKKHVWFG | peptide (Type |
| | ESMDGGFQFSYGNPELPEDVLDVQLAFLRLLSSRASQNITYHCKNSIAYMDQAS | III) |
| | GNVKKALKLMGSNEGEFKAEGNSKFTYTVLEDGCTKHTGEWSKTVFEYRTRKAV | |
| | RLPIVDIAPYDIGGPDQEFGVDVGPVCFL | |
| 37 | RSEPMDFKINTDEIMTSLKSVNGQIESLISPDGSRKNPARNCRDLKFCHPELKS | Trimerization |
| | GEYWVDPNQGCKLDAIKVFCNMETGETCISANPLNVPRKHWWTDSSAEKKHVWF | peptide (Type |
| | GESMDGGFQFSYGNPELPEDVLDVQLAFLRLLSSRASQNITYHCKNSIAYMDQA | III) |
| | SGNVKKALKLMGSNEGEFKAEGNSKFTYTVLEDGCTKHTGEWSKTVFEYRTRKA | |
| | VRLPIVDIAPYDIGGPDQEFGVDVGPVCFL | |
| 38 | ANVVRDRDLEVDTTLKSLSQQIENIRSPEGSRKNPARTCRDLKMCHSDWKSGEY | Trimerization |
| | WIDPNQGCNLDAIKVFCNMETGETCVYPTQPSVAQKNWYISKNPKDKRHVWFGE | peptide (Type |
| | SMTDGFQFEYGGQGSDPADVAIQLTFLRLMSTEASQNITYHCKNSVAYMDQQTG | I), QT version |
| | NLKKALLLQGSNEIEIRAEGNSRFTYSVTVDGCTSHTGAWGKTVIEYKTTKTSR | |
| | LPIIDVAPLDVGAPDQEFGFDVGPVCFL | |
| 39 | SDANVVRDRDLEVDTTLKSLSQQIENIRSPEGSRKNPARTCRDLKMCHSDWKSG | Trimerization |
| | EYWIDPNQGCNLDAIKVFCNMETGETCVYPTQPSVAQKNWYISKNPKDKRHVWF | peptide (Type |
| | GESMTDGFQFEYGGQGSDPADVAIQLTFLRLMSTEASQNITYHCKNSVAYMDQQ | I), QT version |
| | TGNLKKALLLQGSNEIEIRAEGNSRFTYSVTVDGCTSHTGAWGKTVIEYKTTKT | |
| | SRLPIIDVAPLDVGAPDQEFGFDVGPVCFL | |
| 40 | RSDANVVRDRDLEVDTTLKSLSQQIENIRSPEGSRKNPARTCRDLKMCHSDWKS | Trimerization |
| | GEYWIDPNQGCNLDAIKVFCNMETGETCVYPTQPSVAQKNWYISKNPKDKRHVW | peptide (Type |
| | FGESMTDGFQFEYGGQGSDPADVAIQLTFLRLMSTEASQNITYHCKNSVAYMDQ | I), QT version |
| | QTGNLKKALLLQGSNEIEIRAEGNSRFTYSVTVDGCTSHTGAWGKTVIEYKTTK | |
| | TSRLPIIDVAPLDVGAPDQEFGFDVGPVCFL | |
| 41 | GSDANVVRDRDLEVDTTLKSLSQQIENIRSPEGSRKNPARTCRDLKMCHSDWKS | Trimerization |
| | GEYWIDPNQGCNLDAIKVFCNMETGETCVYPTQPSVAQKNWYISKNPKDKRHVW | peptide (Type |
| | FGESMTDGFQFEYGGQGSDPADVAIQLTFLRLMSTEASQNITYHCKNSVAYMDQ | I), QT version |
| | QTGNLKKALLLQGSNEIEIRAEGNSRFTYSVTVDGCTSHTGAWGKTVIEYKTTK | |
| | TSRLPIIDVAPLDVGAPDQEFGFDVGPVCFL | |
| 42 | RVEPKSCDKTHTCPPCPAPELLGGPSVFLFPPKPKDTLMISRTPEVTCVVVDVS | Fc |
| | HEDPEVKFNWYVDGVEVHNAKTKPREEQYNSTYRVVSVLTVLHQDWLNGKEYKC | |
| | KVSNKALPAPIEKTISKAKGQPREPQVYTLPPSRDELTKNQVSLTCLVKGFYPS | |
| | DIAVEWESNGQPENNYKTTPPVLDSDGSFFLYSKLTVDKSRWQQGNVFSCSVMH | |
| | EALHNHYTQKSLSLSPGK | |
| 43 | MGPGRPAPAPWPRHLLRCVLLLGCLHLGRPGAPGDAALPEPNVFLIFSHGLQGC | Full length |
| | LEAQGGQVRVTPACNTSLPAQRWKWVSRNRLFNLGTMQCLGTGWPGTNTTASLG | Endo180-Fc (D1- |
| | MYECDREALNLRWHCRTLGDQLSLLLGARTSNISKPGTLERGDQTRSGQWRIYG | 10-Fc) |
| | SEEDLCALPYHEVYTIQGNSHGKPCTIPFKYDNQWFHGCTSTGREDGHLWCATT | |
| | QDYGKDERWGFCPIKSNDCETFWDKDQLTDSCYQFNFQSTLSWREAWASCEQQG | |
| | ADLLSITEIHEQTYINGLLTGYSSTLWIGLNDLDTSGGWQWSDNSPLKYLNWES | |
| | DQPDNPSEENCGVIRTESSGGWQNRDCSIALPYVCKKKPNATAEPTPPDRWANV | |
| | KVECEPSWQPFQGHCYRLQAEKRSWQESKKACLRGGGDLVSIHSMAELEFITKQ | |
| | IKQEVEELWIGLNDLKLQMNFEWSDGSLVSFTHWHPFEPNNERDSLEDCVTIWG | |
| | PEGRWNDSPCNQSLPSICKKAGQLSQGAAEEDHGCRKGWTWHSPSCYWLGEDQV | |
| | TYSEARRLCTDHGSQLVTITNRFEQAFVSSLIYNWEGEYFWTALQDLNSTGSFF | |
| | WLSGDEVMYTHWNRDQPGYSRGGCVALATGSAMGLWEVKNCTSFRARYICRQSL | |
| | GTPVTPELPGPDPTPSLTGSCPQGWASDTKLRYCYKVESSERLQDKKSWVQAQG | |
| | ACQELGAQLLSLASYEEEHFVANMLNKIFGESEPEIHEQHWFWIGLNRRDPRGG | |
| | QSWRWSDGVGFSYHNFDRSRHDDDDIRGCAVLDLASLQWVAMQCDTQLDWICKI | |
| | PRGTDVREPDDSPQGRREWLRFQEAEYKFFEHHSTWAQAQRICTWFQAELTSVH | |
| | SQAELDFLSHNLQKFSRAQEQHWWIGLHTSESDGRFRWTDGSIINFISWAPGKP | |
| | RPVGKDKKCVYMTASREDWGDQRCLTALPYICKRSNVTKETQPPDLPTTALGGC | |
| | PSDWIQFLNKCFQVQGQEPQSRVKWSEAQFSCEQQEAQLVTITNPLEQAFITAS | |
| | LPNVTFDLWIGLHASQRDFQWVEQEPLMYANWAPGEPSGPSPAPSGNKPTSCAV | |
| | VLHSPSAHFTGRWDDRSCTEETHGFICQKGTDPSLSPSPAALPPAPGTELSYLN | |
| | GTFRLLQKPLRWHDALLLCESRNASLAYVPDPYTQAFLTQAARGLRTPLWIGLA | |
| | GEEGSRRYSWVSEEPLNYVGWQDGEPQQPGGCTYVDVDGAWRTTSCDTKLQGAV | |
| | CGVSSGPPPPRRISYHGSCPQGLADSAWIPFREHCYSFHMELLLGHKEARQRCQ | |
| | RAGGAVLSILDEMENVFVWEHLQSYEGQSRGAWLGMNFNPKGGTLVWQDNTAVN | |
| | YSNWGPPGLGPSMLSHNSCYWIQSNSGLWRPGACTNITMGVVCKRVEPKSCDKT | |
| | HTCPPCPAPELLGGPSVFLFPPKPKDTLMISRTPEVTCVVVDVSHEDPEVKENW | |
| | YVDGVEVHNAKTKPREEQYNSTYRVVSVLTVLHQDWLNGKEYKCKVSNKALPAP | |
| | IEKTISKAKGQPREPQVYTLPPSRDELTKNQVSLTCLVKGFYPSDIAVEWESNG | |
| | QPENNYKTTPPVLDSDGSFFLYSKLTVDKSRWQQGNVFSCSVMHEALHNHYTQK | |
| | SLSLSPGK | |
| 44 | GAPGDAALPEPNVFLIFSHGLQGCLEAQGGQVRVTPACNTSLPAQRWKWVSRNR | Mature Endo-180- |
| | LFNLGTMQCLGTGWPGTNTTASLGMYECDREALNLRWHCRTLGDQLSLLLGART | Fc (D1-10-Fc) |
| | SNISKPGTLERGDQTRSGQWRIYGSEEDLCALPYHEVYTIQGNSHGKPCTIPFK | |
| | YDNQWFHGCTSTGREDGHLWCATTQDYGKDERWGFCPIKSNDCETFWDKDQLTD | |
| | SCYQFNFQSTLSWREAWASCEQQGADLLSITEIHEQTYINGLLTGYSSTLWIGL | |
| | NDLDTSGGWQWSDNSPLKYLNWESDQPDNPSEENCGVIRTESSGGWQNRDCSIA | |
| | LPYVCKKKPNATAEPTPPDRWANVKVECEPSWQPFQGHCYRLQAEKRSWQESKK | |
| | ACLRGGGDLVSIHSMAELEFITKQIKQEVEELWIGLNDLKLQMNFEWSDGSLVS | |
| | FTHWHPFEPNNERDSLEDCVTIWGPEGRWNDSPCNQSLPSICKKAGQLSQGAAE | |
| | EDHGCRKGWTWHSPSCYWLGEDQVTYSEARRLCTDHGSQLVTITNRFEQAFVSS | |
| | LIYNWEGEYFWTALQDLNSTGSFFWLSGDEVMYTHWNRDQPGYSRGGCVALATG | |
| | SAMGLWEVKNCTSFRARYICRQSLGTPVTPELPGPDPTPSLTGSCPQGWASDTK | |
| | LRYCYKVFSSERLQDKKSWVQAQGACQELGAQLLSLASYEEEHFVANMLNKIFG | |
| | ESEPEIHEQHWFWIGLNRRDPRGGQSWRWSDGVGFSYHNFDRSRHDDDDIRGCA | |
| | VLDLASLQWVAMQCDTQLDWICKIPRGTDVREPDDSPQGRREWLRFQEAEYKFF | |
| | EHHSTWAQAQRICTWFQAELTSVHSQAELDFLSHNLQKFSRAQEQHWWIGLHTS | |
| | ESDGRFRWTDGSIINFISWAPGKPRPVGKDKKCVYMTASREDWGDQRCLTALPY | |
| | ICKRSNVTKETQPPDLPTTALGGCPSDWIQFLNKCFQVQGQEPQSRVKWSEAQF | |
| | SCEQQEAQLVTITNPLEQAFITASLPNVTFDLWIGLHASQRDFQWVEQEPLMYA | |
| | NWAPGEPSGPSPAPSGNKPTSCAVVLHSPSAHFTGRWDDRSCTEETHGFICQKG | |
| | TDPSLSPSPAALPPAPGTELSYLNGTFRLLQKPLRWHDALLLCESRNASLAYVP | |
| | DPYTQAFLTQAARGLRTPLWIGLAGEEGSRRYSWVSEEPLNYVGWQDGEPQQPG | |
| | GCTYVDVDGAWRTTSCDTKLQGAVCGVSSGPPPPRRISYHGSCPQGLADSAWIP | |
| | FREHCYSFHMELLLGHKEARQRCQRAGGAVLSILDEMENVFVWEHLQSYEGQSR | |
| | GAWLGMNFNPKGGTLVWQDNTAVNYSNWGPPGLGPSMLSHNSCYWIQSNSGLWR | |
| | PGACTNITMGVVCKRVEPKSCDKTHTCPPCPAPELLGGPSVFLFPPKPKDTLMI | |
| | SRTPEVTCVVVDVSHEDPEVKFNWYVDGVEVHNAKTKPREEQYNSTYRVVSVLT | |
| | VLHQDWLNGKEYKCKVSNKALPAPIEKTISKAKGQPREPQVYTLPPSRDELTKN | |
| | QVSLTCLVKGFYPSDIAVEWESNGQPENNYKTTPPVLDSDGSFFLYSKLTVDKS | |
| | RWQQGNVFSCSVMHEALHNHYTQKSLSLSPGK | |
| 45 | MGPGRPAPAPWPRHLLRCVLLLGCLHLGRPRSPVTPELPGPDPTPSLTGSCPQG | Full Length D6- |
| | WASDTKLRYCYKVFSSERLQDKKSWVQAQGACQELGAQLLSLASYEEEHFVANM | Fc |
| | LNKIFGESEPEIHEQHWFWIGLNRRDPRGGQSWRWSDGVGFSYHNFDRSRHDDD | |
| | DIRGCAVLDLASLQWVAMQCDTQLDWICKIPRVEPKSCDKTHTCPPCPAPELLG | |
| | GPSVFLFPPKPKDTLMISRTPEVTCVVVDVSHEDPEVKENWYVDGVEVHNAKTK | |
| | PREEQYNSTYRVVSVLTVLHQDWLNGKEYKCKVSNKALPAPIEKTISKAKGQPR | |
| | EPQVYTLPPSRDELTKNQVSLTCLVKGFYPSDIAVEWESNGQPENNYKTTPPVL | |
| | DSDGSFFLYSKLTVDKSRWQQGNVFSCSVMHEALHNHYTQKSLSLSPGK | |
| 46 | RSPVTPELPGPDPTPSLTGSCPQGWASDTKLRYCYKVFSSERLQDKKSWVQAQG | Mature D6-Fc |
| | ACQELGAQLLSLASYEEEHFVANMLNKIFGESEPEIHEQHWFWIGLNRRDPRGG | |
| | QSWRWSDGVGFSYHNFDRSRHDDDDIRGCAVLDLASLQWVAMQCDTQLDWICKI | |
| | PRVEPKSCDKTHTCPPCPAPELLGGPSVFLFPPKPKDTLMISRTPEVTCVVVDV | |
| | SHEDPEVKFNWYVDGVEVHNAKTKPREEQYNSTYRVVSVLTVLHQDWLNGKEYK | |
| | CKVSNKALPAPIEKTISKAKGQPREPQVYTLPPSRDELTKNQVSLTCLVKGFYP | |
| | SDIAVEWESNGQPENNYKTTPPVLDSDGSFFLYSKLTVDKSRWQQGNVFSCSVM | |
| | HEALHNHYTQKSLSLSPGK | |

1. A method for purifying a trimeric fusion protein from a sample, comprising:
(a) contacting a sample comprising a trimeric fusion protein with a soluble Endo180 polypeptide linked to a support,
wherein the trimeric fusion protein comprises a heterologous polypeptide sequence fused to a C-terminal polypeptide sequence of a procollagen, wherein the C-terminal polypeptide sequences form inter-polypeptide disulfide bonds, and,
wherein the soluble Endo180 polypeptide specifically binds to the C-terminal polypeptide sequence and/or trimer thereof, whereby the trimeric fusion protein and the soluble Endo180 polypeptide form a complex on the support;
(b) removing unbound and/or nonspecifically bound molecules from the complex; and
(c) dissociating the complex to release the trimeric fusion protein, thereby purifying the trimeric fusion protein from the sample.

2-5. (canceled)

6. The method of claim 1, wherein the trimeric fusion protein is encoded by a recombinant polynucleotide sequence comprising a polynucleotide sequence encoding the heterologous polypeptide sequence fused in-frame to a polynucleotide sequence encoding the C-terminal polypeptide sequence of the procollagen.

7-9. (canceled)

10. The method of claim 1, wherein the soluble Endo180 polypeptide is a fusion polypeptide, or an Endo180 Fc fusion protein.

11. (canceled)

12. The method of claim 1, wherein the support comprises a resin, a particle, a bead, a solid substrate, a membrane, or a combination thereof.

13. The method of claim 1, wherein the removing comprises washing the complex with a buffer.

14. (canceled)

15. The method of claim 1, wherein the dissociating comprises eluting the trimeric fusion protein from the complex with a buffer and the eluting comprises using a plurality of buffers having increasing salt concentrations.

16. The method of claim 1, wherein the soluble Endo180 polypeptide is immobilized on the support via being covalently linked to the support or non-covalently linked to the support.

17-18. (canceled)

19. The method of claim 16, wherein the soluble Endo180 polypeptide comprises an Fc region, wherein the soluble Endo180 polypeptide is non-covalently linked to a Protein A resin via specific binding of Protein A to the Fc region.

20. The method of claim 1, wherein the soluble Endo180 polypeptide comprises an ecto-domain of Endo180.

21. The method of claim 20, wherein the soluble Endo180 polypeptide comprises Domains 1-10 (D1-10) of Endo180.

22. The method of claim 21, wherein the soluble Endo180 polypeptide is D1-10-Fc.

23. The method of claim 20, wherein the soluble Endo180 polypeptide comprises a sequence at least 70%, at least 75%, at least 80%, at least 85%, at least 90%, at least 95%, at least 99%, or 100% identical to SEQ ID NO: 1, SEQ ID NO: 2, SEQ ID NO: 43, or SEQ ID NO: 44, or a fragment or variant thereof.

24. The method of claim 20, wherein the soluble Endo180 polypeptide comprises domains D1, D1-3, D1-4, D1-5, D1-6, and/or D1-7 of Endo180.

25. The method of claim 24, wherein the soluble Endo180 polypeptide is D6-10, D6-9, D6-8, D6-7, or D6 of Endo180, and/or Fc thereof.

26-31. (canceled)

32. The method of claim 25, wherein the soluble Endo180 polypeptide comprises a sequence at least 70%, at least 75%, at least 80%, at least 85%, at least 90%, at least 95%, at least 99%, or 100% identical to SEQ ID NO: 3, SEQ ID NO: 4, SEQ ID NO: 45, or SEQ ID NO: 46, or a fragment or variant thereof.

33-51. (canceled)

52. The method of claim 1, wherein the heterologous polypeptide sequence comprises a TRAIL protein or fragment or variant thereof, a 4-1-BBL protein or fragment or variant thereof, an OX40L protein or fragment or variant thereof, or RNA virus

53-56. (canceled)

57. The method of claim 52, wherein the RNA virus is selected from the group consisting of an HIV, an RSV, a coronavirus, an influenza virus, and a rabies virus.

58. (canceled)

59. The method of claim 57, wherein the coronavirus is a SARS-CoV-2.

60-67. (canceled)

68. The method of claim 1, wherein the C-terminal polypeptide sequence is human.

69. The method of claim 1, wherein the procollagen is proα1(I), proα1(II), proα1(II), proα1(V), proα1(XI), proα2(I), proα2(V), proα2(XI), or proα3(XI), or a fragment or variant thereof.

70-114. (canceled)


SEQUENCE LISTING (46 sequences)

SEQ ID NO: 1 - protein, 1632 aa, Artificial Sequence
  Full length Endo180-Fc (D1-10-Fc)
MGPGRPAPAPWPRHLLRCVLLLGCLHLGRPGAPGDAALPEPNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDLCALPYHEVYTIQGNSHGKPCTIPFKYDNQWFHGCTSTGREDGHLWCATTQDYGKDERWGFCPIKSNDCETFWDKDQLTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCKKKPNATAEPTPPDRWANVKVECEPSWQPFQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICKKAGQLSQGAAEEDHGCRKGWTWHSPSCYWLGEDQVTYSEARRLCTDHGSQLVTITNRFEQAFVSSLIYNWEGEYFWTALQDLNSTGSFFWLSGDEVMYTHWNRDQPGYSRGGCVALATGSAMGLWEVKNCTSFRARYICRQSLGTPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRGTDVREPDDSPQGRREWLRFQEAEYKFFEHHSTWAQAQRICTWFQAELTSVHSQAELDFLSHNLQKFSRAQEQHWWIGLHTSESDGRFRWTDGSIINFISWAPGKPRPVGKDKKCVYMTASREDWGDQRCLTALPYICKRSNVTKETQPPDLPTTALGGCPSDWIQFLNKCFQVQGQEPQSRVKWSEAQFSCEQQEAQLVTITNPLEQAFITASLPNVTFDLWIGLHASQRDFQWVEQEPLMYANWAPGEPSGPSPAPSGNKPTSCAVVLHSPSAHFTGRWDDRSCTEETHGFICQKGTDPSLSPSPAALPPAPGTELSYLNGTFRLLQKPLRWHDALLLCESRNASLAYVPDPYTQAFLTQAARGLRTPLWIGLAGEEGSRRYSWVSEEPLNYVGWQDGEPQQPGGCTYVDVDGAWRTTSCDTKLQGAVCGVSSGPPPPRRISYHGSCPQGLADSAWIPFREHCYSFHMELLLGHKEARQRCQRAGGAVLSILDEMENVFVWEHLQSYEGQSRGAWLGMNFNPKGGTLVWQDNTAVNYSNWGPPGLGPSMLSHNSCYWIQSNSGLWRPGACTNITMGVVCKQAYVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 1602 aa, Artificial Sequence
  Mature Endo-180-Fc(D1-10-Fc)
GAPGDAALPEPNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDLCALPYHEVYTIQGNSHGKPCTIPFKYDNQWFHGCTSTGREDGHLWCATTQDYGKDERWGFCPIKSNDCETFWDKDQLTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCKKKPNATAEPTPPDRWANVKVECEPSWQPFQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICKKAGQLSQGAAEEDHGCRKGWTWHSPSCYWLGEDQVTYSEARRLCTDHGSQLVTITNRFEQAFVSSLIYNWEGEYFWTALQDLNSTGSFFWLSGDEVMYTHWNRDQPGYSRGGCVALATGSAMGLWEVKNCTSFRARYICRQSLGTPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRGTDVREPDDSPQGRREWLRFQEAEYKFFEHHSTWAQAQRICTWFQAELTSVHSQAELDFLSHNLQKFSRAQEQHWWIGLHTSESDGRFRWTDGSIINFISWAPGKPRPVGKDKKCVYMTASREDWGDQRCLTALPYICKRSNVTKETQPPDLPTTALGGCPSDWIQFLNKCFQVQGQEPQSRVKWSEAQFSCEQQEAQLVTITNPLEQAFITASLPNVTFDLWIGLHASQRDFQWVEQEPLMYANWAPGEPSGPSPAPSGNKPTSCAVVLHSPSAHFTGRWDDRSCTEETHGFICQKGTDPSLSPSPAALPPAPGTELSYLNGTFRLLQKPLRWHDALLLCESRNASLAYVPDPYTQAFLTQAARGLRTPLWIGLAGEEGSRRYSWVSEEPLNYVGWQDGEPQQPGGCTYVDVDGAWRTTSCDTKLQGAVCGVSSGPPPPRRISYHGSCPQGLADSAWIPFREHCYSFHMELLLGHKEARQRCQRAGGAVLSILDEMENVFVWEHLQSYEGQSRGAWLGMNFNPKGGTLVWQDNTAVNYSNWGPPGLGPSMLSHNSCYWIQSNSGLWRPGACTNITMGVVCKQAYVRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 427 aa, Artificial Sequence
  Full Length D6-Fc
MGPGRPAPAPWPRHLLRCVLLLGCLHLGRPRSPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 397 aa, Artificial Sequence
  Mature D6-Fc
RSPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 30 aa, Artificial Sequence
  Signal Peptide
MGPGRPAPAPWPRHLLRCVLLLGCLHLGRP

SEQ ID NO: 6 - protein, 232 aa, Artificial Sequence
  Fc
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 1384 aa, Artificial Sequence
  Mature Endo180 Ecto-domain (Uniprot Q9UBG0)
GAPGDAALPEPNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDLCALPYHEVYTIQGNSHGKPCTIPFKYDNQWFHGCTSTGREDGHLWCATTQDYGKDERWGFCPIKSNDCETFWDKDQLTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCKKKPNATAEPTPPDRWANVKVECEPSWQPFQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICKKAGQLSQGAAEEDHGCRKGWTWHSPSCYWLGEDQVTYSEARRLCTDHGSQLVTITNRFEQAFVSSLIYNWEGEYFWTALQDLNSTGSFFWLSGDEVMYTHWNRDQPGYSRGGCVALATGSAMGLWEVKNCTSFRARYICRQSLGTPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRGTDVREPDDSPQGRREWLRFQEAEYKFFEHHSTWAQAQRICTWFQAELTSVHSQAELDFLSHNLQKFSRAQEQHWWIGLHTSESDGRFRWTDGSIINFISWAPGKPRPVGKDKKCVYMTASREDWGDQRCLTALPYICKRSNVTKETQPPDLPTTALGGCPSDWIQFLNKCFQVQGQEPQSRVKWSEAQFSCEQQEAQLVTITNPLEQAFITASLPNVTFDLWIGLHASQRDFQWVEQEPLMYANWAPGEPSGPSPAPSGNKPTSCAVVLHSPSAHFTGRWDDRSCTEETHGFICQKGTDPSLSPSPAALPPAPGTELSYLNGTFRLLQKPLRWHDALLLCESRNASLAYVPDPYTQAFLTQAARGLRTPLWIGLAGEEGSRRYSWVSEEPLNYVGWQDGEPQQPGGCTYVDVDGAWRTTSCDTKLQGAVCGVSSGPPPPRRISYHGSCPQGLADSAWIPFREHCYSFHMELLLGHKEARQRCQRAGGAVLSILDEMENVFVWEHLQSYEGQSRGAWLGMNFNPKGGTLVWQDNTAVNYSNWGPPGLGPSMLSHNSCYWIQSNSGLWRPGACTNITMGVVCKLPRAEQSSFSPSALPENPAA

SEQ ID NO: 8 - protein, 127 aa, Artificial Sequence
  D1 (Ricin B-type lectin domain)
PNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDL

SEQ ID NO: 9 - protein, 49 aa, Artificial Sequence
  D2 (Fibronectin type-II domain)
SHGKPCTIPFKYDNQWFHGCTSTGREDGHLWCATTQDYGKDERWGFCPI

SEQ ID NO: 10 - protein, 117 aa, Artificial Sequence
  D3 (C-type lectin 1)
LTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCK

SEQ ID NO: 11 - protein, 117 aa, Artificial Sequence
  D4 (C-type lectin 2)
FQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICK

SEQ ID NO: 12 - protein, 117 aa, Artificial Sequence
  D5 (C-type lectin 3)
HSPSCYWLGEDQVTYSEARRLCTDHGSQLVTITNRFEQAFVSSLIYNWEGEYFWTALQDLNSTGSFFWLSGDEVMYTHWNRDQPGYSRGGCVALATGSAMGLWEVKNCTSFRARYIC

SEQ ID NO: 13 - protein, 132 aa, Artificial Sequence
  D6 (C-type lectin 4)
KLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICK

SEQ ID NO: 14 - protein, 120 aa, Artificial Sequence
  D7 (C-type lectin 5)
FQEAEYKFFEHHSTWAQAQRICTWFQAELTSVHSQAELDFLSHNLQKFSRAQEQHWWIGLHTSESDGRFRWTDGSIINFISWAPGKPRPVGKDKKCVYMTASREDWGDQRCLTALPYICK

SEQ ID NO: 15 - protein, 129 aa, Artificial Sequence
  D8 (C-type lectin 6)
FLNKCFQVQGQEPQSRVKWSEAQFSCEQQEAQLVTITNPLEQAFITASLPNVTFDLWIGLHASQRDFQWVEQEPLMYANWAPGEPSGPSPAPSGNKPTSCAVVLHSPSAHFTGRWDDRSCTEETHGFIC

SEQ ID NO: 16 - protein, 112 aa, Artificial Sequence
  D9 (C-type lectin 7)
YLNGTFRLLQKPLRWHDALLLCESRNASLAYVPDPYTQAFLTQAARGLRTPLWIGLAGEEGSRRYSWVSEEPLNYVGWQDGEPQQPGGCTYVDVDGAWRTTSCDTKLQGAVC

SEQ ID NO: 17 - protein, 121 aa, Artificial Sequence
  D10 (C-type lectin 8)
FREHCYSFHMELLLGHKEARQRCQRAGGAVLSILDEMENVFVWEHLQSYEGQSRGAWLGMNFNPKGGTLVWQDNTAVNYSNWGPPGLGPSMLSHNSCYWIQSNSGLWRPGACTNITMGVVC

SEQ ID NO: 18 - protein, 309 aa, Artificial Sequence
  Trimerization peptide (Type I) with glycine- X-Y repeats and D-N mutation at BMP-1 site, KS version
NGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 19 - protein, 309 aa, Artificial Sequence
  Trimerization peptide (Type I) with glycine-X-Y repeats and A-N mutation at BMP-1 site, KS version
NGLPGPIGPPGPRGRTGDAGPVGPPGPPGPPGPPGPPSAGFDFSFLPQPPQEKAHDGGRYYRNDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 20 - protein, 311 aa, Artificial Sequence
  Trimerization peptide (Type I) with glycine-X-Y repeats and D-N mutation at BMP-1 site, KS version
RSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 21 - protein, 311 aa, Artificial Sequence
  Trimerization peptide (Type I) with glycine-X-Y repeats and D-N mutation at BMP-1 site, KS version
GSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 22 - protein, 309 aa, Artificial Sequence
  Trimerization peptide (Type I) with glycine-X-Y repeats, no BMP-1 site mutation, KS version
NGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 23 - protein, 310 aa, Artificial Sequence
  Trimerization peptide (Type I) with glycine-X-Y repeats, no BMP-1 site mutation, KS version
SNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 24 - protein, 309 aa, Artificial Sequence
  Trimerization peptide (Type I) with glycine-X-Y repeats, no BMP-1 site mutation, QT version
NGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 25 - protein, 310 aa, Artificial Sequence
  Trimerization peptide (Type I) with glycine-X-Y repeats, no BMP-1 site mutation, QT version
SNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 26 - protein, 244 aa, Artificial Sequence
  Trimerization peptide (Type I), KS version
ANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 27 - protein, 244 aa, Artificial Sequence
  Trimerization peptide (Type I), KS version
DANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCF

SEQ ID NO: 28 - protein, 246 aa, Artificial Sequence
  Trimerization peptide (Type I), KS version
SDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 29 - protein, 247 aa, Artificial Sequence
  Trimerization peptide (Type I), KS version
RSDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 30 - protein, 309 aa, Artificial Sequence
  Trimerization peptide (Type I), with glycine-X-Y repeats and D-N mutation at BMP-1 site, QT version
NGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 31 - protein, 309 aa, Artificial Sequence
  Trimerization peptide (Type I), with glycine-X-Y repeats and A-N mutation at BMP-1 site, QT version
NGLPGPIGPPGPRGRTGDAGPVGPPGPPGPPGPPGPPSAGFDFSFLPQPPQEKAHDGGRYYRNDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 32 - protein, 311 aa, Artificial Sequence
  Trimerization peptide (Type I), with glycine-X-Y repeats and D-N mutation at BMP-1 site, QT version
RSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 33 - protein, 311 aa, Artificial Sequence
  Trimerization peptide (Type I), with glycine-X-Y repeats and D-N mutation at BMP-1 site, QT version
GSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 34 - protein, 234 aa, Artificial Sequence
  Trimerization peptide (Type III)
DEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCF

SEQ ID NO: 35 - protein, 244 aa, Artificial Sequence
  Trimerization peptide (Type III)
EPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 36 - protein, 245 aa, Artificial Sequence
  Trimerization peptide (Type III)
SEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 37 - protein, 246 aa, Artificial Sequence
  Trimerization peptide (Type III)
RSEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 38 - protein, 244 aa, Artificial Sequence
  Trimerization peptide (Type I), QT version
ANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 39 - protein, 246 aa, Artificial Sequence
  Trimerization peptide (Type I), QT version
SDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 40 - protein, 247 aa, Artificial Sequence
  Trimerization peptide (Type I), QT version
RSDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 41 - protein, 247 aa, Artificial Sequence
  Trimerization peptide (Type I), QT version
GSDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 42 - protein, 234 aa, Artificial Sequence
  Fc
RVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 1628 aa, Artificial Sequence
  Full length Endo180-Fc (D1-10-Fc)
MGPGRPAPAPWPRHLLRCVLLLGCLHLGRPGAPGDAALPEPNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDLCALPYHEVYTIQGNSHGKPCTIPFKYDNQWFHGCTSTGREDGHLWCATTQDYGKDERWGFCPIKSNDCETFWDKDQLTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCKKKPNATAEPTPPDRWANVKVECEPSWQPFQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICKKAGQLSQGAAEEDHGCRKGWTWHSPSCYWLGEDQVTYSEARRLCTDHGSQLVTITNRFEQAFVSSLIYNWEGEYFWTALQDLNSTGSFFWLSGDEVMYTHWNRDQPGYSRGGCVALATGSAMGLWEVKNCTSFRARYICRQSLGTPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRGTDVREPDDSPQGRREWLRFQEAEYKFFEHHSTWAQAQRICTWFQAELTSVHSQAELDFLSHNLQKFSRAQEQHWWIGLHTSESDGRFRWTDGSIINFISWAPGKPRPVGKDKKCVYMTASREDWGDQRCLTALPYICKRSNVTKETQPPDLPTTALGGCPSDWIQFLNKCFQVQGQEPQSRVKWSEAQFSCEQQEAQLVTITNPLEQAFITASLPNVTFDLWIGLHASQRDFQWVEQEPLMYANWAPGEPSGPSPAPSGNKPTSCAVVLHSPSAHFTGRWDDRSCTEETHGFICQKGTDPSLSPSPAALPPAPGTELSYLNGTFRLLQKPLRWHDALLLCESRNASLAYVPDPYTQAFLTQAARGLRTPLWIGLAGEEGSRRYSWVSEEPLNYVGWQDGEPQQPGGCTYVDVDGAWRTTSCDTKLQGAVCGVSSGPPPPRRISYHGSCPQGLADSAWIPFREHCYSFHMELLLGHKEARQRCQRAGGAVLSILDEMENVFVWEHLQSYEGQSRGAWLGMNFNPKGGTLVWQDNTAVNYSNWGPPGLGPSMLSHNSCYWIQSNSGLWRPGACTNITMGVVCKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 1598 aa, Artificial Sequence
  Mature Endo-180-Fc(D1-10-Fc)
GAPGDAALPEPNVFLIFSHGLQGCLEAQGGQVRVTPACNTSLPAQRWKWVSRNRLFNLGTMQCLGTGWPGTNTTASLGMYECDREALNLRWHCRTLGDQLSLLLGARTSNISKPGTLERGDQTRSGQWRIYGSEEDLCALPYHEVYTIQGNSHGKPCTIPFKYDNQWFHGCTSTGREDGHLWCATTQDYGKDERWGFCPIKSNDCETFWDKDQLTDSCYQFNFQSTLSWREAWASCEQQGADLLSITEIHEQTYINGLLTGYSSTLWIGLNDLDTSGGWQWSDNSPLKYLNWESDQPDNPSEENCGVIRTESSGGWQNRDCSIALPYVCKKKPNATAEPTPPDRWANVKVECEPSWQPFQGHCYRLQAEKRSWQESKKACLRGGGDLVSIHSMAELEFITKQIKQEVEELWIGLNDLKLQMNFEWSDGSLVSFTHWHPFEPNNFRDSLEDCVTIWGPEGRWNDSPCNQSLPSICKKAGQLSQGAAEEDHGCRKGWTWHSPSCYWLGEDQVTYSEARRLCTDHGSQLVTITNRFEQAFVSSLIYNWEGEYFWTALQDLNSTGSFFWLSGDEVMYTHWNRDQPGYSRGGCVALATGSAMGLWEVKNCTSFRARYICRQSLGTPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRGTDVREPDDSPQGRREWLRFQEAEYKFFEHHSTWAQAQRICTWFQAELTSVHSQAELDFLSHNLQKFSRAQEQHWWIGLHTSESDGRFRWTDGSIINFISWAPGKPRPVGKDKKCVYMTASREDWGDQRCLTALPYICKRSNVTKETQPPDLPTTALGGCPSDWIQFLNKCFQVQGQEPQSRVKWSEAQFSCEQQEAQLVTITNPLEQAFITASLPNVTFDLWIGLHASQRDFQWVEQEPLMYANWAPGEPSGPSPAPSGNKPTSCAVVLHSPSAHFTGRWDDRSCTEETHGFICQKGTDPSLSPSPAALPPAPGTELSYLNGTFRLLQKPLRWHDALLLCESRNASLAYVPDPYTQAFLTQAARGLRTPLWIGLAGEEGSRRYSWVSEEPLNYVGWQDGEPQQPGGCTYVDVDGAWRTTSCDTKLQGAVCGVSSGPPPPRRISYHGSCPQGLADSAWIPFREHCYSFHMELLLGHKEARQRCQRAGGAVLSILDEMENVFVWEHLQSYEGQSRGAWLGMNFNPKGGTLVWQDNTAVNYSNWGPPGLGPSMLSHNSCYWIQSNSGLWRPGACTNITMGVVCKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 427 aa, Artificial Sequence
  Full Length D6-Fc
MGPGRPAPAPWPRHLLRCVLLLGCLHLGRPRSPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 397 aa, Artificial Sequence
  Mature D6-Fc
RSPVTPELPGPDPTPSLTGSCPQGWASDTKLRYCYKVFSSERLQDKKSWVQAQGACQELGAQLLSLASYEEEHFVANMLNKIFGESEPEIHEQHWFWIGLNRRDPRGGQSWRWSDGVGFSYHNFDRSRHDDDDIRGCAVLDLASLQWVAMQCDTQLDWICKIPRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK